SEQUENCE LISTING (10 sequences)

SEQ ID NO: 1 - DNA, 9678 bp, Hepatitis C virus
  misc_feature: JFH1
acctgcccctaataggggcgacactccgccatgaatcactcccctgtgaggaactactgtcttcacgcagaaagcgcctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggcgcttgcgagtgccccgggaggtctcgtagaccgtgcaccatgagcacaaatcctaaacctcaaagaaaaaccaaaagaaacaccaaccgtcgcccagaagacgttaagttcccgggcggcggccagatcgttggcggagtatacttgttgccgcgcaggggccccaggttgggtgtgcgcacgacaaggaaaacttcggagcggtcccagccacgtgggagacgccagcccatccccaaagatcggcgctccactggcaaggcctggggaaaaccaggtcgcccctggcccctatatgggaatgagggactcggctgggcaggatggctcctgtccccccgaggctctcgcccctcctggggccccactgacccccggcataggtcgcgcaacgtgggtaaagtcatcgacaccctaacgtgtggctttgccgacctcatggggtacatccccgtcgtaggcgccccgcttagtggcgccgccagagctgtcgcgcacggcgtgagagtcctggaggacggggttaattatgcaacagggaacctacccggtttccccttttctatcttcttgctggccctgttgtcctgcatcaccgttccggtctctgctgcccaggtgaagaataccagtagcagctacatggtgaccaatgactgctccaatgacagcatcacttggcagctcgaggctgcggttctccacgtccccgggtgcgtcccgtgcgagagagtggggaatacgtcacggtgttgggtgccagtctcgccaaacatggctgtgcggcagcccggtgccctcacgcagggtctgcggacgcacatcgatatggttgtgatgtccgccaccttctgctctgctctctacgtgggggacctctgtggcggggtgatgctcgcggcccaggtgttcatcgtctcgccgcagtaccactggtttgtgcaagaatgcaattgctccatctaccctggcaccatcactggacaccgcatggcatgggacatgatgatgaactggtcgcccacggccaccatgatcctggcgtacgtgatgcgcgtccccgaggtcatcatagacatcgttagcggggctcactggggcgtcatgttcggcttggcctacttctctatgcagggagcgtgggcgaaggtcattgtcatccttctgctggccgctggggtggacgcgggcaccaccaccgttggaggcgctgttgcacgttccaccaacgtgattgccggcgtgttcagccatggccctcagcagaacattcagctcattaacaccaacggcagttggcacatcaaccgtactgccttgaattgcaatgactccttgaacaccggctttctcgcggccttgttctacaccaaccgctttaactcgtcagggtgtccagggcgcctgtccgcctgccgcaacatcgaggctttccggatagggtggggcaccctacagtacgaggataatgtcaccaatccagaggatatgaggccgtactgctggcactaccccccaaagccgtgtggcgtagtccccgcgaggtctgtgtgtggcccagtgtactgtttcacccccagcccggtagtagtgggcacgaccgacagacgtggagtgcccacctacacatggggagagaatgagacagatgtcttcctactgaacagcacccgaccgccgcagggctcatggttcggctgcacgtggatgaactccactggtttcaccaagacttgtggcgcgccaccttgccgcaccagagctgacttcaacgccagcacggacttgttgtgccctacggattgttttaggaagcatcctgatgccacttatattaagtgtggttctgggccctggctcacaccaaagtgcctggtccactacccttacagactctggcattacccctgcacagtcaattttaccatcttcaagataagaatgtatgtagggggggttgagcacaggctcacggccgcatgcaacttcactcgtggggatcgctgcgacttggaggacagggacaggagtcagctgtctcctctgttgcactctaccacggaatgggccatcctgccctgcacctactcagacttacccgctttgtcaactggtcttctccaccttcaccagaacatcgtggacgtacaatacatgtatggcctctcacctgctatcacaaaatacgtcgttcgatgggagtgggtggtactcttattcctgctcttagcggacgccagagtctgcgcctgcttgtggatgctcatcttgttgggccaggccgaagcagcattggagaagttggtcgtcttgcacgctgcgagtgcggctaactgccatggcctcctatattttgccatcttcttcgtggcagcttggcacatcaggggtcgggtggtccccttgaccacctattgcctcactggcctatggcccttctgcctactgctcatggcactgccccggcaggcttatgcctatgacgcacctgtgcacggacagataggcgtgggtttgttgatattgatcaccctcttcacactcaccccggggtataagaccctcctcggccagtgtctgtggtggttgtgctatctcctgaccctgggggaagccatgattcaggagtgggtaccacccatgcaggtgcgcggcggccgcgatggcatcgcgtgggccgtcactatattctgcccgggtgtggtgtttgacattaccaaatggcttttggcgttgcttgggcctgcttacctcttaagggccgctttgacacatgtgccgtacttcgtcagagctcacgctctgataagggtatgcgctttggtgaagcagctcgcggggggtaggtatgttcaggtggcgctattggcccttggcaggtggactggcacctacatctatgaccacctcacacctatgtcggactgggccgctagcggcctgcgcgacttagcggtcgccgtggaacccatcatcttcagtccgatggagaagaaggtcatcgtctggggagcggagacggctgcatgtggggacattctacatggacttcccgtgtccgcccgactcggccaggagatcctcctcggcccagctgatggctacacctccaaggggtggaagctccttgctcccatcactgcttatgcccagcaaacacgaggcctcctgggcgccatagtggtgagtatgacggggcgtgacaggacagaacaggccggggaagtccaaatcctgtccacagtctctcagtccttcctcggaacaaccatctcgggggttttgtggactgtttaccacggagctggcaacaagactctagccggcttacggggtccggtcacgcagatgtactcgagtgctgagggggacttggtaggctggcccagcccccctgggaccaagtctttggagccgtgcaagtgtggagccgtcgacctatatctggtcacgcggaacgctgatgtcatcccggctcggagacgcggggacaagcggggagcattgctctccccgagacccatttcgaccttgaaggggtcctcgggggggccggtgctctgccctaggggccacgtcgttgggctcttccgagcagctgtgtgctctcggggcgtggccaaatccatcgatttcatccccgttgagacactcgacgttgttacaaggtctcccactttcagtgacaacagcacgccaccggctgtgccccagacctatcaggtcgggtacttgcatgctccaactggcagtggaaagagcaccaaggtccctgtcgcgtatgccgcccaggggtacaaagtactagtgcttaacccctcggtagctgccaccctggggtttggggcgtacctatccaaggcacatggcatcaatcccaacattaggactggagtcaggaccgtgatgaccggggaggccatcacgtactccacatatggcaaatttctcgccgatgggggctgcgctagcggcgcctatgacatcatcatatgcgatgaatgccacgctgtggatgctacctccattctcggcatcggaacggtccttgatcaagcagagacagccggggtcagactaactgtgctggctacggccacaccccccgggtcagtgacaaccccccatcccgatatagaagaggtaggcctcgggcgggagggtgagatccccttctatgggagggcgattcccctatcctgcatcaagggagggagacacctgattttctgccactcaaagaaaaagtgtgacgagctcgcggcggcccttcggggcatgggcttgaatgccgtggcatactatagagggttggacgtctccataataccagctcagggagatgtggtggtcgtcgccaccgacgccctcatgacggggtacactggagactttgactccgtgatcgactgcaatgtagcggtcacccaagctgtcgacttcagcctggaccccaccttcactataaccacacagactgtcccacaagacgctgtctcacgcagtcagcgccgcgggcgcacaggtagaggaagacagggcacttataggtatgtttccactggtgaacgagcctcaggaatgtttgacagtgtagtgctttgtgagtgctacgacgcaggggctgcgtggtacgatctcacaccagcggagaccaccgtcaggcttagagcgtatttcaacacgcccggcctacccgtgtgtcaagaccatcttgaattttgggaggcagttttcaccggcctcacacacatagacgcccacttcctctcccaaacaaagcaagcgggggagaacttcgcgtacctagtagcctaccaagctacggtgtgcgccagagccaaggcccctcccccgtcctgggacgccatgtggaagtgcctggcccgactcaagcctacgcttgcgggccccacacctctcctgtaccgtttgggccctattaccaatgaggtcaccctcacacaccctgggacgaagtacatcgccacatgcatgcaagctgaccttgaggtcatgaccagcacgtgggtcctagctggaggagtcctggcagccgtcgccgcatattgcctggcgactggatgcgtttccatcatcggccgcttgcacgtcaaccagcgagtcgtcgttgcgccggataaggaggtcctgtatgaggcttttgatgagatggaggaatgcgcctctagggcggctctcatcgaagaggggcagcggatagccgagatgttgaagtccaagatccaaggcttgctgcagcaggcctctaagcaggcccaggacatacaacccgctatgcaggcttcatggcccaaagtggaacaattttgggccagacacatgtggaacttcattagcggcatccaatacctcgcaggattgtcaacactgccagggaaccccgcggtggcttccatgatggcattcagtgccgccctcaccagtccgttgtcgaccagtaccaccatccttctcaacatcatgggaggctggttagcgtcccagatcgcaccacccgcgggggccaccggctttgtcgtcagtggcctggtgggggctgccgtgggcagcataggcctgggtaaggtgctggtggacatcctggcaggatatggtgcgggcatttcgggggccctcgtcgcattcaagatcatgtctggcgagaagccctctatggaagatgtcatcaatctactgcctgggatcctgtctccgggagccctggtggtgggggtcatctgcgcggccattctgcgccgccacgtgggaccgggggagggcgcggtccaatggatgaacaggcttattgcctttgcttccagaggaaaccacgtcgcccctactcactacgtgacggagtcggatgcgtcgcagcgtgtgacccaactacttggctctcttactataaccagcctactcagaagactccacaattggataactgaggactgccccatcccatgctccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacaggactgaccactgacaatctgaaaattccttgccaactaccttctccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaagttgccaatcaaccctttgagtaactcgctgttgcgataccataacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcggtagagcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgt

SEQ ID NO: 2 - DNA, 9683 bp, Artificial Sequence
  synthetic DNA J6/JFH1
acccgcccctaataggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggtgcttgcgagtgccccgggaggtctcgtagaccgtgcaccatgagcacaaatcctaaacctcaaagaaaaaccaaaagaaacaccaaccgtcgcccacaagacgttaagtttccgggcggcggccagatcgttggcggagtatacttgttgccgcgcaggggccccaggttgggtgtgcgcgcgacaaggaagacttcggagcggtcccagccacgtggaaggcgccagcccatccctaaagatcggcgctccactggcaaatcctggggaaaaccaggatacccctggcccctatacgggaatgagggactcggctgggcaggatggctcctgtccccccgaggttcccgtccctcttggggccccaatgacccccggcataggtcgcgcaacgtgggtaaggtcatcgataccctaacgtgcggctttgccgacctcatggggtacatccctgtcgtgggcgccccgctcggcggcgtcgccagagctctcgcgcatggcgtgagagtcctggaggacggggttaattttgcaacagggaacttacccggttgctccttttctatcttcttgctggccctgctgtcctgcatcaccaccccggtctccgctgccgaagtgaagaacatcagtaccggctacatggtgactaacgactgcaccaatgacagcattacctggcagctccaggctgctgtcctccacgtccccgggtgcgtcccgtgcgagaaagtggggaatgcatctcagtgctggataccggtctcaccgaatgtggccgtgcagcggcccggcgccctcacgcagggcttgcggacgcacatcgacatggttgtgatgtccgccacgctctgctctgccctctacgtgggggacctctgcggtggggtgatgctcgcagcccaaatgttcattgtctcgccgcagcaccactggtttgtccaagactgcaattgctccatctaccctggtaccatcactggacaccgcatggcatgggacatgatgatgaactggtcgcccacggctaccatgatcttggcgtacgcgatgcgtgtccccgaggtcattatagacatcattagcggggctcattggggcgtcatgttcggcttggcctacttctctatgcagggagcgtgggcgaaagtcgttgtcatccttctgttggccgccggggtggacgcgcgcacccatactgttgggggttctgccgcgcagaccaccgggcgcctcaccagcttatttgacatgggccccaggcagaaaatccagctcgttaacaccaatggcagctggcacatcaaccgcaccgccctgaactgcaatgactccttgcacaccggctttatcgcgtctctgttctacacccacagcttcaactcgtcaggatgtcccgaacgcatgtccgcctgccgcagtatcgaggccttccgggtgggatggggcgccttgcaatatgaggataatgtcaccaatccagaggatatgagaccctattgctggcactacccaccaaggcagtgtggcgtggtctccgcgaagactgtgtgtggcccagtgtactgtttcacccccagcccagtggtagtgggcacgaccgacaggcttggagcgcccacttacacgtggggggagaatgagacagatgtcttcctattgaacagcactcgaccaccgctggggtcatggttcggctgcacgtggatgaactcttctggctacaccaagacttgcggcgcaccaccctgccgtactagagctgacttcaacgccagcacggacctgttgtgccccacggactgttttaggaagcatcctgataccacttacctcaaatgcggctctgggccctggctcacgccaaggtgcctgatcgactacccctacaggctctggcattacccctgcacagttaactataccatcttcaaaataaggatgtatgtgggaggggttgagcacaggctcacggctgcatgcaatttcactcgtggggatcgttgcaacttggaggacagagacagaagtcaactgtctcctttgttgcactccaccacggaatgggccattttaccttgctcttactcggacctgcccgccttgtcgactggtcttctccacctccaccaaaacatcgtggacgtacaattcatgtatggcctatcacctgccctcacaaaatacatcgtccgatgggagtgggtaatactcttattcctgctcttagcggacgccagggtttgcgcctgcttatggatgctcatcttgttgggccaggccgaagcagcactagagaagctggtcatcttgcacgctgcgagcgcagctagctgcaatggcttcctatattttgtcatctttttcgtggctgcttggtacatcaagggtcgggtagtccccttagctacctattccctcactggcctgtggtcctttagcctactgctcctagcattgccccaacaggcttatgcttatgacgcatctgtgcatggccagataggagcggctctgctggtaatgatcaccctcttcacactcaccccggggtataagaccctcctcggccagtgtctgtggtggttgtgctatctcctgaccctgggggaagccatgattcaggagtgggtaccacccatgcaggtgcgcggcggccgcgatggcatcgcgtgggccgtcactatattctgcccgggtgtggtgtttgacattaccaaatggcttttggcgttgcttgggcctgcttacctcttaagggccgctttgacacatgtgccgtacttcgtcagagctcacgctctgataagggtatgcgctttggtgaagcagctcgcggggggtaggtatgttcaggtggcgctattggcccttggcaggtggactggcacctacatctatgaccacctcacacctatgtcggactgggccgctagcggcctgcgcgacttagcggtcgccgtggaacccatcatcttcagtccgatggagaagaaggtcatcgtctggggagcggagacggctgcatgtggggacattctacatggacttcccgtgtccgcccgactcggccaggagatcctcctcggcccagctgatggctacacctccaaggggtggaagctccttgctcccatcactgcttatgcccagcaaacacgaggcctcctgggcgccatagtggtgagtatgacggggcgtgacaggacagaacaggccggggaagtccaaatcctgtccacagtctctcagtccttcctcggaacaaccatctcgggggttttgtggactgtttaccacggagctggcaacaagactctagccggcttacggggtccggtcacgcagatgtactcgagtgctgagggggacttggtaggctggcccagcccccctgggaccaagtctttggagccgtgcaagtgtggagccgtcgacctatatctggtcacgcggaacgctgatgtcatcccggctcggagacgcggggacaagcggggagcattgctctccccgagacccatttcgaccttgaaggggtcctcgggggggccggtgctctgccctaggggccacgtcgttgggctcttccgagcagctgtgtgctctcggggcgtggccaaatccatcgatttcatccccgttgagacactcgacgttgttacaaggtctcccactttcagtgacaacagcacgccaccggctgtgccccagacctatcaggtcgggtacttgcatgctccaactggcagtggaaagagcaccaaggtccctgtcgcgtatgccgcccaggggtacaaagtactagtgcttaacccctcggtagctgccaccctggggtttggggcgtacctatccaaggcacatggcatcaatcccaacattaggactggagtcaggaccgtgatgaccggggaggccatcacgtactccacatatggcaaatttctcgccgatgggggctgcgctagcggcgcctatgacatcatcatatgcgatgaatgccacgctgtggatgctacctccattctcggcatcggaacggtccttgatcaagcagagacagccggggtcagactaactgtgctggctacggccacaccccccgggtcagtgacaaccccccatcccgatatagaagaggtaggcctcgggcgggagggtgagatccccttctatgggagggcgattcccctatcctgcatcaagggagggagacacctgattttctgccactcaaagaaaaagtgtgacgagctcgcggcggcccttcggggcatgggcttgaatgccgtggcatactatagagggttggacgtctccataataccagctcagggagatgtggtggtcgtcgccaccgacgccctcatgacggggtacactggagactttgactccgtgatcgactgcaatgtagcggtcacccaagctgtcgacttcagcctggaccccaccttcactataaccacacagactgtcccacaagacgctgtctcacgcagtcagcgccgcgggcgcacaggtagaggaagacagggcacttataggtatgtttccactggtgaacgagcctcaggaatgtttgacagtgtagtgctttgtgagtgctacgacgcaggggctgcgtggtacgatctcacaccagcggagaccaccgtcaggcttagagcgtatttcaacacgcccggcctacccgtgtgtcaagaccatcttgaattttgggaggcagttttcaccggcctcacacacatagacgcccacttcctctcccaaacaaagcaagcgggggagaacttcgcgtacctagtagcctaccaagctacggtgtgcgccagagccaaggcccctcccccgtcctgggacgccatgtggaagtgcctggcccgactcaagcctacgcttgcgggccccacacctctcctgtaccgtttgggccctattaccaatgaggtcaccctcacacaccctgggacgaagtacatcgccacatgcatgcaagctgaccttgaggtcatgaccagcacgtgggtcctagctggaggagtcctggcagccgtcgccgcatattgcctggcgactggatgcgtttccatcatcggccgcttgcacgtcaaccagcgagtcgtcgttgcgccggataaggaggtcctgtatgaggcttttgatgagatggaggaatgcgcctctagggcggctctcatcgaagaggggcagcggatagccgagatgttgaagtccaagatccaaggcttgctgcagcaggcctctaagcaggcccaggacatacaacccgctatgcaggcttcatggcccaaagtggaacaattttgggccagacacatgtggaacttcattagcggcatccaatacctcgcaggattgtcaacactgccagggaaccccgcggtggcttccatgatggcattcagtgccgccctcaccagtccgttgtcgaccagtaccaccatccttctcaacatcatgggaggctggttagcgtcccagatcgcaccacccgcgggggccaccggctttgtcgtcagtggcctggtgggggctgccgtgggcagcataggcctgggtaaggtgctggtggacatcctggcaggatatggtgcgggcatttcgggggccctcgtcgcattcaagatcatgtctggcgagaagccctctatggaagatgtcatcaatctactgcctgggatcctgtctccgggagccctggtggtgggggtcatctgcgcggccattctgcgccgccacgtgggaccgggggagggcgcggtccaatggatgaacaggcttattgcctttgcttccagaggaaaccacgtcgcccctactcactacgtgacggagtcggatgcgtcgcagcgtgtgacccaactacttggctctcttactataaccagcctactcagaagactccacaattggataactgaggactgccccatcccatgctccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacaggactgaccactgacaatctgaaaattccttgccaactaccttctccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaagttgccaatcaaccctttgagtaactcgctgttgcgataccataacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcggtagagcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgtctaga

SEQ ID NO: 3 - DNA, 9669 bp, Artificial Sequence
  synthetic DNA TH/JFH1
acctgcccctaataggggcgacactccgccatgaatcactcccctgtgaggaactactgtcttcacgcagaaagcgcctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggcgcttgcgagtgccccgggaggtctcgtagaccgtgcaccatgagcacgaatcctaaacctcaaagaaaaaccaaacgtaacaccaaccgccgcccacaggacgtcaagttcccgggcggtggccagatcgttggtggagtttacctgttgccgcgcaggggccccaggttgggtgtgcgcgcgactaggaagacttccgagcggtcgcaacctcgtggaaggcgacaacctatccccaaggatcgccgacccgagggcagggcctgggctcagcctgggtacccttggcccctctatggcaacgagggcatggggtgggcaggatggctcctgtcaccccgtggctcccggcctagttggggccccaatgacccccggcgcaggtcgcgtaatttgggtaaagtcatcgatacccttacatgcggcttcgccgacctcatggggtacattccgctcgtcggcgctcccttggggggcgctgccagggccttggcgcatggcgtccgggttctggaggacggcgtgaactatgcaacagggaatctgcccggttgctctttctctatcttcctcttggctctgctgtcctgtctaaccatcccagcttccgcttatgaagtgcgcaacgtgtccggggtgtaccatgtcacgaacgactgctccaactcgagcattgtgtacgagacaggggacatgattatgcacacccctgggtgcgtgccctgtgttcgggagaacaactcctcccgctgctgggcagcgctcactcccacgctcgcggccaggaacgccagcgtccccaccacgacaatacggcgccacgtcgatttgctcgttggggcggctgctttctgctccgctatgtacgtgggggatctctgcggatctgttttcctcgtctcccagttgttcaccttctcgcctcgccggcatgagacagtgcaggactgcaattgttcaatctatcccggccacgtatcaggtcaccgcatggcttgggatatgatgatgaactggtcacctacaacagccctactggtatcgcagttactccggatcccacaagccgtcgtggacatggtggcgggggcccactggggagtcctggcgggccttgcctactattccatggcggggaactgggctaaggttttgattgtgctgctactctttgccggcgttgatggggcgacctacgtgacgggggggtcggaagccagaggggcctctggcttagcaaacctcttttcatttggggcgtctcagaagatccagctcataaataccaacggcagttggcacatcaatagaactgccctgaactgcaatgactccctccacactgggtttcttgccgcgctattctacacacacaaattcaacgcgtccggatgtccagagcgcatggccagctgccgccccattgaagagttcgctcaggggtatggtcccatcacttatgctgagccctccccctcggaccagaggccctattgctggcactacgcgcctcgaccgtgtggtatcatacccgcgtcgcaggtgtgtggtccagtgtactgcttcaccccaagccctgttgtggtggggacgaccgatcgctccggtgcccccacgtataattggggggcgaatgagacggacgtgctgtatctcaacaacacgcggccgccgcaaggcaactggttcggctgcacatggatgaatggcaccgggttcaccaagacgtgcgggggccccccgtgcaacatcggggggggcggcaacaacaacaccttgacctgccccacggactgtttccggaaacaccccgaggccacctacaccaaatgtggttcgggaccttggttgacacctaggtgcatggtcgactacccatacaggctctggcactacccctgcaccgttaactttaccatctttaaggttaggatgtacgtgggaggtgtggagcacaggctcaacgccgcatgcaattggacccgaggagagcgttgtaacttagaggacagggatagatcagagcttagcccgctgctgctgtcaacaacagagtggcaggtgctaccttgttccttcaccaccctaccggctctgtccactggtttgatccatctccaccagaacatcgtggacgtgcaatacctgtacggtatagggtcggcggttgtctcctatgcaatcaaatgggaatatgtcttgttgctcttcctcctcctggcagacgcgcgcgtctgcgcctgcttgtggatgatgctgctgatagctcaagctgaggccgccttagagaacctggtggtcctcaatgcggcgtccctggctggagcgcatggccttctctctttccttgtgttcttctgtgccgcttggtacatcaagggcaggttgatccccggggcggcgtatgctttttacggcgtatggccgctgctcctactcctgctggcgttaccaccacgagcatacgccatggaccgggagatggctgcatcgtgcggaggcgcggtttttgtaggtctggcattcctgaccttgtcaccacactataaggcattcctcgccaagctcctgtggtggttgtgctatctcctgaccctgggggaagccatgattcaggagtgggtaccacccatgcaggtgcgcggcggccgcgatggcatcgcgtgggccgtcactatattctgcccgggtgtggtgtttgacattaccaaatggcttttggcgttgcttgggcctgcttacctcttaagggccgctttgacacatgtgccgtacttcgtcagagctcacgctctgataagggtatgcgctttggtgaagcagctcgcggggggtaggtatgttcaggtggcgctattggcccttggcaggtggactggcacctacatctatgaccacctcacacctatgtcggactgggccgctagcggcctgcgcgacttagcggtcgccgtggaacccatcatcttcagtccgatggagaagaaggtcatcgtctggggagcggagacggctgcatgtggggacattctacatggacttcccgtgtccgcccgactcggccaggagatcctcctcggcccagctgatggctacacctccaaggggtggaagctccttgctcccatcactgcttatgcccagcaaacacgaggcctcctgggcgccatagtggtgagtatgacggggcgtgacaggacagaacaggccggggaagtccaaatcctgtccacagtctctcagtccttcctcggaacaaccatctcgggggttttgtggactgtttaccacggagctggcaacaagactctagccggcttacggggtccggtcacgcagatgtactcgagtgctgagggggacttggtaggctggcccagcccccctgggaccaagtctttggagccgtgcaagtgtggagccgtcgacctatatctggtcacgcggaacgctgatgtcatcccggctcggagacgcggggacaagcggggagcattgctctccccgagacccatttcgaccttgaaggggtcctcgggggggccggtgctctgccctaggggccacgtcgttgggctcttccgagcagctgtgtgctctcggggcgtggccaaatccatcgatttcatccccgttgagacactcgacgttgttacaaggtctcccactttcagtgacaacagcacgccaccggctgtgccccagacctatcaggtcgggtacttgcatgctccaactggcagtggaaagagcaccaaggtccctgtcgcgtatgccgcccaggggtacaaagtactagtgcttaacccctcggtagctgccaccctggggtttggggcgtacctatccaaggcacatggcatcaatcccaacattaggactggagtcaggaccgtgatgaccggggaggccatcacgtactccacatatggcaaatttctcgccgatgggggctgcgctagcggcgcctatgacatcatcatatgcgatgaatgccacgctgtggatgctacctccattctcggcatcggaacggtccttgatcaagcagagacagccggggtcagactaactgtgctggctacggccacaccccccgggtcagtgacaaccccccatcccgatatagaagaggtaggcctcgggcgggagggtgagatccccttctatgggagggcgattcccctatcctgcatcaagggagggagacacctgattttctgccactcaaagaaaaagtgtgacgagctcgcggcggcccttcggggcatgggcttgaatgccgtggcatactatagagggttggacgtctccataataccagctcagggagatgtggtggtcgtcgccaccgacgccctcatgacggggtacactggagactttgactccgtgatcgactgcaatgtagcggtcacccaagctgtcgacttcagcctggaccccaccttcactataaccacacagactgtcccacaagacgctgtctcacgcagtcagcgccgcgggcgcacaggtagaggaagacagggcacttataggtatgtttccactggtgaacgagcctcaggaatgtttgacagtgtagtgctttgtgagtgctacgacgcaggggctgcgtggtacgatctcacaccagcggagaccaccgtcaggcttagagcgtatttcaacacgcccggcctacccgtgtgtcaagaccatcttgaattttgggaggcagttttcaccggcctcacacacatagacgcccacttcctctcccaaacaaagcaagcgggggagaacttcgcgtacctagtagcctaccaagctacggtgtgcgccagagccaaggcccctcccccgtcctgggacgccatgtggaagtgcctggcccgactcaagcctacgcttgcgggccccacacctctcctgtaccgtttgggccctattaccaatgaggtcaccctcacacaccctgggacgaagtacatcgccacatgcatgcaagctgaccttgaggtcatgaccagcacgtgggtcctagctggaggagtcctggcagccgtcgccgcatattgcctggcgactggatgcgtttccatcatcggccgcttgcacgtcaaccagcgagtcgtcgttgcgccggataaggaggtcctgtatgaggcttttgatgagatggaggaatgcgcctctagggcggctctcatcgaagaggggcagcggatagccgagatgttgaagtccaagatccaaggcttgctgcagcaggcctctaagcaggcccaggacatacaacccgctatgcaggcttcatggcccaaagtggaacaattttgggccagacacatgtggaacttcattagcggcatccaatacctcgcaggattgtcaacactgccagggaaccccgcggtggcttccatgatggcattcagtgccgccctcaccagtccgttgtcgaccagtaccaccatccttctcaacatcatgggaggctggttagcgtcccagatcgcaccacccgcgggggccaccggctttgtcgtcagtggcctggtgggggctgccgtgggcagcataggcctgggtaaggtgctggtggacatcctggcaggatatggtgcgggcatttcgggggccctcgtcgcattcaagatcatgtctggcgagaagccctctatggaagatgtcatcaatctactgcctgggatcctgtctccgggagccctggtggtgggggtcatctgcgcggccattctgcgccgccacgtgggaccgggggagggcgcggtccaatggatgaacaggcttattgcctttgcttccagaggaaaccacgtcgcccctactcactacgtgacggagtcggatgcgtcgcagcgtgtgacccaactacttggctctcttactataaccagcctactcagaagactccacaattggataactgaggactgccccatcccatgctccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacaggactgaccactgacaatctgaaaattccttgccaactaccttctccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaagttgccaatcaaccctttgagtaactcgctgttgcgataccataacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcggtagagcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgt

SEQ ID NO: 4 - DNA, 21 bp, Artificial Sequence
  synthetic oligonucleotide Mod87
gggggggcgacgatcgtcagg

SEQ ID NO: 5 - DNA, 21 bp, Artificial Sequence
  synthetic oligonucleotide Mod87s
nnnnnnncgacgatcgtcang

SEQ ID NO: 6 - DNA, 28 bp, Artificial Sequence
  Synthetic primer J6E2Fc-s
cacaagcttcgcacccatactgttgggg

SEQ ID NO: 7 - DNA, 30 bp, Artificial Sequence
  Synthetic primer J6E2Fc-as
acaggatcccatcggacgatgtattttgtg

SEQ ID NO: 8 - DNA, 32 bp, Artificial Sequence
  Synthetic primer J6E2dTM-as
gctctagattaccatcggacgatgtattttgt

SEQ ID NO: 9 - DNA, 30 bp, Artificial Sequence
  Synthetic primer J6E1F-s
cacaagcttgccgaagtgaagaacatcagt

SEQ ID NO: 10 - DNA, 32 bp, Artificial Sequence
  Synthetic primer J6E1F-as
tcgggatcccaatgagccccgctaatgatgtc

(19) United States
(12) Patent Application Publication
(10) Pub. No.: US 2012/0251572 A1
(43) Pub. Date: Oct. 4, 2012

(54) HEPATITIS C VIRUS VACCINE COMPOSITION

(76) Inventors: Takaji Wakita, Tokyo (JP); Masaki Moriyama, Kanagawa (JP); Daisuke Akazawa, Kanagawa (JP); Noriko Nakamura, Kanagawa (JP)

(21) Appl. No.: 13/499,009

(22) PCT Filed: Sep. 30, 2010
(86) PCT No.: PCT/JP2010/067096

§ 371 Date: Mar. 29, 2012

(30) Foreign Application Priority Data
Sep. 30, 2009 (JP) 2009-228145

(51) Int. Cl. A61K 39/29; A61P 37/04

(52) U.S. Cl. 424/228.1

(57) ABSTRACT

A combination of an HCV antigen for inducing an antibody having the activity of inhibiting HCV infection and an optimum adjuvant is discovered, so as to provide an effective HCV vaccine composition. The hepatitis C virus vaccine composition comprises: inactivated viral particles obtained by inactivating infectious hepatitis C virus particles prepared from hepatitis C virus genome that contains sequences encoding NS3 protein, NS4A protein, NS4B protein, NS5A protein and NS5B protein derived from hepatitis C virus JFH1 strain;
an oligonucleotide containing unmethylated CpG, according to SEQ ID NO: 5 in the sequence listing; and
aluminium hydroxide.

### TECHNICAL FIELD

The present invention relates to a hepatitis C virus vaccine composition.

### BACKGROUND ART

The hepatitis C virus (which may be abbreviated as “HCV” hereinafter) was discovered as a major causative virus of non-A and non-B hepatitis (non-patent document 1). HCV is a single-stranded (+) RNA virus having a genome length of approximately 9.6 kb, in which the genome encodes a precursor protein that is divided into 10 types of virus protein (i.e., Core, E1, E2, p7, NS2, NS3, NS4A, NS4B, NS5A, and NS5B proteins) via post-translational cleavage by signal peptidase from host or proteases from HCV. Of these virus proteins, Core, E1, E2, and p7 proteins are classified as structural proteins, and NS2, NS3, NS4A, NS4B, NS5A, and NS5B proteins are classified as non-structural proteins. HCV is also classified into 10 or more genotypes (e.g., 1a, 1b, 2a, 2b, 3a, and 3b) depending on differences in the nucleotide sequences of the genomes (Non-patent documents 2 to 4). Genotypes can be determined by an HCV antibody test, an HCV core antigen test, or a nucleic acid amplification test.

HCV is transmitted from human to human via blood, causing chronic hepatitis among about 60% to 80% of infected persons. When hepatitis is left without any appropriate treatment, it is known to cause cirrhosis or liver cancer within about 20 to 30 years after infection. Therefore, early detection of HCV infection for prevention of the onset of chronic hepatitis and early treatment of the disease after the onset thereof are desired.

Generally, a major therapeutic method for hepatitis C entails administration of interferon alone or combination therapy with interferon and ribavirin. However, it has been recently revealed that the therapeutic effects of interferons differ significantly depending on differences in HCV genotypes, and the effects are not easily exhibited on HCV of genotype 1a or 1b (non-patent documents 5 and 6). It has also been revealed that the intensity of the antiviral action of interferons varies even between HCV of genotype 2a and HCV of genotype 2b, upon which the relatively good effects of interferon are observed. It has been suggested that interferons exhibit antiviral action more strongly against HCV of genotype 2a than against HCV of genotype 2b (non-patent document 7).

Hence, the development of an HCV vaccine that makes it possible to prevent the onset of chronic hepatitis in virus carriers who have not yet developed chronic hepatitis after infection with HCV or to enhance autoimmunity after the onset of chronic hepatitis, so as to eliminate HCV, is desired.

However, both obtainment of HCV particles with infectivity (hereinafter, may be abbreviated as “infectious HCV particles”) and testing of the effects of inhibiting HCV infection are difficult. Moreover, it is difficult to find a combination of an adjuvant and an antigen that enhances humoral immunity and cellular immunity required for exhibition of the effects of inhibiting HCV infection. Hence, no HCV vaccine composition having sufficient activity of inhibiting HCV infection has yet been developed.

In general, adjuvants can be classified based on physical properties into particulate adjuvants and non-particulate adjuvants.

Examples of a particulate adjuvant include aluminum salts, water-in-oil type emulsions, oil-in-water type emulsions, immune-stimulating complexes, liposomes, nanoparticles, and fine particles. A particulate adjuvant alone is often mixed with an antigen and then used. In particular, aluminium hydroxide (hereinafter, may be abbreviated as “Alum”) is used for medicinal use as a low inflammatory adjuvant. However, it is known that a combination of Alum and an antigen having medium to low antigen titer is unable to enhance humoral immunity and cellular immunity as required for exhibition of the effects of inhibiting infection. Thus, Alum is inappropriate for use as an adjuvant.

Meanwhile, examples of a non-particulate adjuvant include a muramyl dipeptide (an active ingredient of an adjuvant of peptide glycan extracted from Mycobacteria), a nonionic block copolymer, saponin (a complex mixture of triterpenoid extracted from Quillaja saponaria bark), lipid A (glycosamine disaccharides with five or six C12-C16 fatty acid chains and 2 phosphate radicals), cytokine, nucleic acid, a carbohydrate polymer, derivatized polysaccharides, and bacterial toxin (e.g., cholera toxin and E. coli thermolabile toxin), which are often used in combination with a particulate adjuvant.

As nucleic acid to be used as an adjuvant, oligodeoxynucleotide containing a CpG dinucleotide motif that has not undergone methylation modification (hereinafter, may be abbreviated as “CpG-ODN”) (non-patent document 8) and double-stranded RNA (hereinafter, may be abbreviated as “dsRNA”) are known.

It has been reported that CpG-ODN containing a CpG motif consisting of a 6-base palindrome motif induces strong cellular cytotoxicity. In particular, 3 sequences (5′-AACGTT-3′, 5′-AGCGCT-3′, and 5′-GACGTC-3′) particularly strongly induce cellular cytotoxicity (non-patent document 9). It has recently been revealed that CpG-ODN is a ligand of TLR9 and induces immune response through activation of TLR9.

Also, a typical example of dsRNA is polyriboinosinic acid-polyribocytidylic acid (hereinafter, referred to as “polyI:C”) prepared by hybridization of polyriboinosinic acid (polyI) with polyribocytidylic acid (polyC). Furthermore, polyICLC, which is a complex of polyI:C, poly L lysine, and carboxymethyl cellulose (non-patent document 10), and polyI:PolyC12U, in which the C portion in polyI:C has been substituted with U (specifically, one of every 12 Cs has been substituted with “U”), are known as less toxic dsRNAs (non-patent document 11). It has been revealed that polyI:C is an inducer of interferon I and a ligand of TLR3.

Also, recombinant proteins (patent documents 1 and 2), synthetic peptides (patent document 3), virus-like particles (patent document 4), naked DNA (patent document 5), and viral particles (patent documents 6 and 7) have been reported as antigen protein candidates to be used for development of an HCV vaccine.

Patent document 1 discloses that antibody titers against the E1 and E2 proteins are increased by mixing the E1 and E2 proteins, which are HCV structural proteins, as antigens with MF59 (submicronic oil-in-water type emulsion) and CpG-ODN as adjuvants, but does not reveal the thus induced antibody's activity of inhibiting HCV infection.

Furthermore, patent document 2 discloses that the production amounts of antibodies against the E1 and E2 proteins are increased by mixing the HCV E1 and E2 proteins as antigens with polyI:C as an adjuvant, but similarly to patent document 1, it does not demonstrate the antibodies' activity of inhibiting HCV infection.

Furthermore, patent documents 6 and 7 describe a vaccine using an HCV particle itself as an antigen, but do not describe any adjuvant, or else merely describe general examples thereof. Moreover, patent documents 6 and 7 never mention the antibody production capacity of the vaccine or the antibody's activity of inhibiting HCV infection.

Furthermore, non-patent document 12 discloses that the production amounts of antibodies against Core, NS3, NS4, and NS5 proteins are increased by mixing HCV Core, NS3, NS4, and NS5 proteins as antigens with Alum and CpG-ODN as adjuvants. However, similarly to patent documents 1 and 2, the document does not disclose the antibodies' activity of inhibiting HCV infection.

### PRIOR ART DOCUMENTS

### Patent documents

Patent document 1 JP Patent Publication (Kohyo) No. 2005-502611 A
Patent document 2 JP Patent No. 4286138
Patent document 3 JP Patent Publication (Kohyo) No. 2009-513542 A
Patent document 4 JP Patent Publication (Kohyo) No. 2001-504337 A
Patent document 5 JP Patent Publication (Kokai) No. 2009-183295 A
Patent document 6 International Patent Publication No. 05/080575
Patent document 7 International Patent Publication No. 06/022422

### Non-Patent Documents

Non-patent document 1 Choo et al., Science, 1989, Vol. 244, p. 359-362
Non-patent document 2 Simmonds et al., Hepatology, 1994, Vol. 10, p. 1321-1324
Non-patent document 3 Okamoto et al., J. Gen. Virol., 1992, Vol. 73, p. 73-679
Non-patent document 4 Mori et al., Biochem. Biophys. Res. Commun., 1992, Vol. 183, p. 334-342
Non-patent document 5 Fried et al., N. Engl. J. Med., 2002, Vol. 347, p. 975-982
Non-patent document 6 Lusida et al., J. Clin. Microbiol., 2001, Vol. 39, p. 3858-3864
Non-patent document 7 Murakami et al., Hepatology, 1999, Vol. 30, p. 1045-1053
Non-patent document 8 Yamamoto et al., Jpn, J. Cancer Res. 1988, Vol. 79, p. 866-873
Non-patent document 9 Yamamoto et al., J. Immunol. 1992, Vol. 148, p. 4072-4076
Non-patent document 10 Nakamura et al., J. Interferon Res. 1982, Vol. 2, p. 1-4
Non-patent document 11 Laurence et al., J. Clin. Invest. 1987, Vol. 80, p. 1631-1639
Non-patent document 12 Vajdy et al., J. Gen. Virol. 2006, Vol. 87, p. 2253-2262

### SUMMARY OF THE INVENTION

### Problem to be Solved by the Invention

An object of the present invention is to provide an effective HCV vaccine composition through discovery of a combination of an HCV antigen for inducing an antibody having activity of inhibiting HCV infection and an optimum adjuvant therefor.

### Means for Solving the Problem

A mouse was immunized with the E2 protein, which is an envelope protein required for HCV particles to adhere to and infect cells, as an antigen, and then antibody titer against the E2 protein in the serum was evaluated. High antibody titer was observed. It has conventionally been believed that antibody titer against the E2 protein correlates with the antibody's activity of inhibiting HCV infection. However, as a result of the study by the present inventors, it has been surprisingly revealed that this hypothesis is not always true. Specifically, as described in the examples of the description, the present inventors demonstrated that when a mouse was inoculated with a vaccine containing Alum and CpG-ODN as adjuvants and the E2 protein, antibody titer against the E2 protein in serum was increased, but the activity of inhibiting HCV infection was not increased.

Hence, to search for a combination of an HCV antigen for inducing an antibody having activity of inhibiting HCV infection and an adjuvant, infectious HCV particles were produced in a cell culture system, purified, and then inactivated, and then vaccine compositions combined with various adjuvants were prepared and then administered to mice. Antibody titer against the HCV E2 protein in serum of each mouse after administration and activity of inhibiting HCV infection were measured. Thus, it was found that the activity of inhibiting HCV infection does not always reach a high level, even if an adjuvant exhibiting high-level antibody titer against the HCV E2 protein is used as described above, but the use of a vaccine composition consisting of inactivated HCV particles+Alum+CpG-ODN results in not only high-level antibody titers against the E1 protein and the E2 protein, but also high-level activity of inhibiting HCV infection. Thus, the present invention was completed. Specifically, the present invention encompasses the following.

The present invention provides a hepatitis C virus vaccine composition, comprising:

inactivated viral particles obtained by inactivating infectious hepatitis C virus particles prepared from the hepatitis C virus genome that contains sequences encoding NS3 protein, NS4A protein, NS4B protein, NS5A protein and NS5B protein derived from the hepatitis C virus JFH1 strain;

an oligonucleotide containing unmethylated CpG shown in SEQ ID NO: 5 in the sequence listing; and

aluminium hydroxide. The infectious hepatitis C virus genome is preferably the hepatitis C virus vaccine composition containing sequences that encode core protein, E1 protein, E2 protein and p7 protein derived from the hepatitis C virus J6CF strain.

The infectious hepatitis C virus genome is also preferably the above hepatitis C virus vaccine composition containing a sequence that encodes 16 amino acid residues from the N-terminal acid residue of NS2 protein derived from the hepatitis C virus J6CF strain and a sequence that encodes a portion ranging from the 17^{th} amino acid residue from the N terminus to the C-terminal amino acid residue of NS2 protein derived from the hepatitis C virus JFH1 strain.

Furthermore, the present invention provides a hepatitis C virus vaccine composition containing:

inactivated viral particles obtained by inactivating infectious hepatitis C virus particles containing NS3 protein, NS4A protein, NS4B protein, NS5A protein and NS5B protein derived from the hepatitis C virus JFH1 strain;

an oligonucleotide containing the unmethylated CpG shown in SEQ ID NO: 5 in the sequence listing; and

aluminium hydroxide. The infectious hepatitis C virus is preferably the above hepatitis C virus vaccine composition containing core protein, E1 protein, E2 protein and p7 protein derived from the hepatitis C virus J6CF strain.

The infectious hepatitis C virus is also preferably the above hepatitis C virus vaccine composition in which the NS2 protein is a chimeric protein, 16 amino acid residues from the N-terminal amino acid residue of the NS2 protein are derived from the J6CF strain, and a portion ranging from the 17th amino acid residue from the N terminus to the C terminal amino acid residue of the NS2 protein is derived from the JFH1 strain.

Furthermore, the present invention provides a hepatitis C virus vaccine composition containing:

inactivated hepatitis C virus particles of genotype 2a;

an oligonucleotide (CpG-ODN) containing the unmethylated CpG shown in SEQ ID NO: 5 in the sequence listing; and

aluminium hydroxide.

The hepatitis C virus particles of genotype 2a preferably have structural proteins of hepatitis C virus of genotype 2a, and particularly structural proteins (core protein, E1 protein, E2 protein, and p7 protein) derived from the J6CF strain. The hepatitis C virus particles of genotype 2a preferably have the E2 protein of hepatitis C virus of genotype 2a and particularly E2 protein derived from the J6CF strain. Furthermore, the hepatitis C virus particles of genotype 2a are preferably viral particles produced by expression of the HCV genome having nucleotide sequences that encode the NS2 protein derived from at least 1 type of hepatitis C virus strain, NS3 protein, NS4A protein, NS4B protein, NS5A protein and NS5B protein derived from the JFH1 strain. The hepatitis C virus particles of genotype 2a are also preferably viral particles produced by expression of a chimeric HCV genome containing nucleotide sequences that encode structural proteins (core protein, E1 protein, E2 protein, and p7 protein) derived from J6CF strain, the NS2 protein that is a chimeric protein derived from the J6CF strain and JFH1 strain (16 amino acid residues from the N-terminal amino acid residue of the NS2 protein are derived from the J6CF strain and a portion ranging from the 17^{th} amino acid residue from the N terminus to the C-terminal amino acid residue of the NS2 protein is derived from the JFH1 strain), and non-structural proteins other than NS2 derived from the JFH1 strain (NS3 protein, NS4A protein, NS4B protein, NS5A protein, and NS5B protein), the 5′ untranslated region, and the 3′ untranslated region.

This description includes the disclosure of the description and/or drawings of Japanese Patent Application No. 2009-228145, from which the present application claims priority.

### Effects of the Invention

The present invention provides a vaccine composition for effectively preventing hepatitis C virus infection.

### BRIEF DESCRIPTION OF THE DRAWINGS

FIG. 1 shows the results of measuring antibody titer against the J6E2 protein by EIA when a J6E2Fc protein (5 μg or 20 μg) as an antigen and Alum or Mod87s alone or in combination as an adjuvant were administered twice to mice at 2 week intervals, serum samples were collected 10 days after the final administration, and then the samples were diluted 1000 fold, 3000 fold, and 10000 fold.

FIG. 2 shows the results of measuring the inhibition activity against J6CF HCVpp infection when a J6E2Fc protein (20 μg) as an antigen and Alum+Mod87s as an adjuvant were administered twice to mice at 2 week intervals, serum samples were collected 10 days after the final administration, and then the serum samples were diluted 100 fold.

FIG. 3 shows the results of measuring antibody titers against the E1 protein when inactivated J6/JFH1-HCV particles (2 pmol) as antigens and Alum, Mod87s, or polyI:C alone or in combination as an adjuvant were administered 4 times to mice at 2 week intervals, serum samples were collected 1 week after the final administration, and then the serum samples were diluted 3000 fold. In FIG. 3, “Saline” denotes the serum of mice to which saline alone was administered.

FIG. 4 shows the results of measuring antibody titers against the E2 protein when inactivated J6/JFH1-HCV particles (2 pmol) as an antigen and Alum, Mod87s or polyI:C alone or in combination as an adjuvant were administered 4 times to mice at 2 week intervals, serum samples were collected 1 week after the final administration, and then the serum samples were diluted 3000 fold. In FIG. 4, “Saline” denotes the serum of mice to which saline alone was administered.

FIG. 5 shows the results of measuring the inhibition activity against J6CF HCVpp (genotype 2a) infection when inactivated J6/JFH1-HCV particles (2 pmol) as an antigen and Alum, Mod87s or polyI:C alone or in combination as an adjuvant were administered 4 times to a mouse at 2 week intervals, serum samples were collected 1 week after the final administration, and then the serum samples were diluted 100 fold. In FIG. 5, “Saline” denotes the serum of a mouse to which saline alone was administered.

FIG. 6 shows the results of measuring the inhibition activity against H77 HCVpp (genotype 1a) infection when inactivated J6/JFH1-HCV particles (2 pmol) as an antigen and Alum, Mod87s or polyI:C alone or in combination as an adjuvant were administered 4 times to mice at 2 week intervals, serum samples were collected 1 week after the final administration, and then the serum samples were diluted 100 fold. In FIG. 6, “Saline” denotes the serum of mice to which saline alone was administered.

FIG. 7 shows the results of measuring the inhibition activity against TH HCVpp (genotype 1b) infection when inactivated J6/JFH1-HCV particles (2 pmol) as an antigen and Alum, Mod87s or polyI:C alone or in combination as an adjuvant were administered 4 times to mice at 2 week intervals, serum samples were collected 1 week after the final administration, and then the serum samples were diluted 100 fold. In FIG. 7, “Saline” denotes the serum of mice to which saline alone was administered.

FIG. 8 shows the results of measuring the inhibition activity against J6/JFH1 HCVcc (genotype 2a) infection when inactivated J6/JFH1-HCV particles (2 pmol) as an antigen and Alum, Mod87s or polyI:C alone or in combination as an adjuvant were administered 4 times to mice at 2 week intervals, serum samples were collected 1 week after the final administration, and then the serum samples were diluted 100 fold. In FIG. 8, “Saline” denotes the serum of mice to which saline alone was administered.

FIG. 9 shows the results of measuring the inhibition activity against TH/JFH1 HCVcc (genotype 1b) infection when inactivated J6/JFH1-HCV particles (2 pmol) as an antigen and Alum, Mod87s or polyI:C alone or in combination as an adjuvant were administered 4 times to mice at 2 week intervals, serum samples were collected 1 week after the final administration, and then the serum samples were diluted 100 fold. In FIG. 9, “Saline” denotes the serum of mice to which saline alone was administered.

### EMBODIMENTS FOR CARRYING OUT THE INVENTION

The present invention will be further described specifically as follows.

The present invention relates to a vaccine composition comprising an adjuvant and an antigen appropriate for inducing an antibody for inhibiting HCV infection, which is produced by inactivating HCV particles (preferably, infectious HCV particles) produced from a specific HCV genome and then using the HCV particle as the antigen.

The present invention can be implemented via conventional molecular biological and immunological techniques within the technical scope in the art. Such techniques are thoroughly described in, for example, Sambrook et al., Molecular Cloning: A Laboratory Manual, Cold Spring Harbor Laboratory (vol. 3, 2001) or Ed Harlow et al., Antibodies: A Laboratory Manual, Cold Spring Harbor Laboratory, 1988.

All publications, patents, and patent applications cited herein are incorporated herein by reference in their entirety.

### (1) Preparation of HCV Particles

A specific example of an HCV genome that can be used for generating infectious HCV particles, which can be used as antigens for the HCV vaccine composition according to the present invention, is viral genome RNA of the JFH1 strain of genotype 2a, which consists of nucleotide sequences of, in order from the 5′ side to the 3′ side, a 5′ untranslated region, a core protein coding region, an E1 protein coding region, an E2 protein coding region, a p7 protein coding region, an NS2 protein coding region, an NS3 protein coding region, an NS4A protein coding region, an NS4B protein coding region, an NS5A protein coding region, an NS5B protein coding region, and a 3′ untranslated region. Alternatively, such an HCV genome may be chimeric RNA consisting of viral genome RNAs of 2 or more types of HCV strain. For example, such a chimeric RNA consists of the nucleotide sequences of, in order from the 5′ side to the 3′ side, a 5′ untranslated region, a core protein coding region, an E1 protein coding region, an E2 protein coding region, and a p7 protein coding region of an HCV strain other than the JFH1 strain, an NS2 protein coding region of an HCV strain other than the JFH1 strain or the JFH1 strain, and the NS3 protein coding region, the NS4A protein coding region, the NS4B protein coding region, the NS5A protein coding region, the NS5B protein coding region, and the 3′ untranslated region of the JFH1 strain. Alternatively, such an HCV genome may be chimeric RNA consisting of nucleotide sequences of, in order from the 5′ side to the 3′ side, a 5′ untranslated region, a core protein coding region, an E1 protein coding region, an E2 protein coding region, and a p7 protein coding region of an HCV strain other than the JFH1 strain, and a chimeric NS2 protein coding region, an NS3 protein coding region, an NS4A protein coding region, an NS4B protein coding region, an NS5A protein coding region, an NS5B protein coding region, and a 3′ untranslated region derived from one or more HCV strains.

Therefore, a preferred embodiment of the HCV genome to be used for preparation of infectious HCV particles that are used for the present invention contains nucleotide sequences encoding at least the NS3 protein, the NS4A protein, the NS4B protein, the NS5A protein, and the NS5B protein, respectively, which are non-structural proteins of the JFH1 strain. Such a virus that has acquired autonomous replication capacity and virus production capacity and contains the full-length or a portion of the genome of the JFH1 strain is referred to as “JFH1-derived virus.”

An example of the HCV genome in a preferred embodiment of the present invention is an HCV genome consisting of, in order from the 5′ side to the 3′ side, a full-length genome derived from the JFH1 strain of genotype 2a (for example, a nucleic acid having the nucleotide sequence shown in SEQ ID NO: 1, provided that nucleotide “T (thymine)” in the nucleotide sequence shown in SEQ ID NO: 1 is read as “U (uracil)” when the genome is RNA. The same applies to other nucleotide sequences in the description). Alternatively, such an HCV genome is a chimeric genome consisting of nucleotide sequences of, in order from the 5′ side to the 3′ side, the 5′ untranslated region, the core protein coding region, the E1 protein coding region, the E2 protein coding region, the p7 protein coding region, and 16 amino acid residues from the N terminal amino acid residue of the NS2 protein coding region derived from the J6CF strain of genotype 2a, and a portion of the NS2 protein coding region which ranges from the 17th amino acid residue from the N terminus to the C terminal amino acid residue, the NS3 protein coding region, the NS4A protein coding region, the NS4B protein coding region, the NS5A protein coding region, the NS5B protein coding region, and the 3′ untranslated region from the JFH1 strain. A particularly preferable example thereof is a nucleic acid cloned into J6/JFH1 consisting of the nucleotide sequence of SEQ ID NO: 2.

In a particularly preferred embodiment of the present invention, the above HCV (chimera) genome is a nucleic acid consisting of the nucleotide sequence shown in SEQ ID NO: 1 or 2. HCV genome RNA or HCV genome DNA can be used for producing the infectious HCV particles of the present invention.

Furthermore, an example of the HCV genome in a preferred embodiment of the present invention is:

a chimeric HCV genome, in which the nucleotide sequences of the 5′ untranslated region from the JFH1 strain, the core protein coding region, the E1 protein coding region, the E2 protein coding region, and the p7 protein coding region from the TH strain of genotype 1b (Wakita, T. et al., J. Biol. Chem., 269, 14205-14210, 1994, JP Patent Publication (Kokai) No. 2004-179 A), and the NS2 protein coding region, the NS3 protein coding region, the NS4A protein coding region, the NS4B protein coding region, the NS5A protein coding region, the NS5B protein coding region, and the 3′ untranslated region from the JFH1 strain are linked in this order; or is preferably

a chimeric HCV genome, in which the 5′ untranslated region derived from the JFH1 strain, the core protein coding region, the E1 protein coding region, the E2 protein coding region, the p7 protein, and a portion of the NS2 protein coding region encoding 33 amino acid residues from the N terminus from the TH strain, and a portion encoding the 34^{th} amino acid residue from the N terminus to the C terminal amino acid residue of the NS2 protein coding region, the NS3 protein coding region, the NS4A protein coding region, the NS4B protein coding region, the NS5A protein coding region, the NS5B protein coding region, and the 3′ untranslated region from the JFH1 strain are linked in this order. A particularly preferable example thereof is a nucleic acid cloned into TH/JFH1 shown in the nucleotide sequence of SEQ ID NO: 3.

In a particularly preferred embodiment of the present invention, the above chimeric HCV genome is a nucleic acid consisting of the nucleotide sequence shown in SEQ ID NO: 3. HCV genome RNA or HCV genome DNA can be used for producing the infectious HCV particles of the present invention.

Specifically, the present invention relates to a vaccine for which antigens are:

HCV particles obtained from a chimeric HCV genome in which the nucleotide sequences of, in order from the 5′ side to the 3′ side, the 5′ untranslated region, the core protein coding region, the E1 protein coding region, the E2 protein coding region, and the p7 protein coding region from the HCV J6CF strain, and the NS2 protein coding region, the NS3 protein coding region, the NS4A protein coding region, the NS4B protein coding region, the NS5A protein coding region, the NS5B protein coding region, and the 3′ untranslated region from the JFH1 strain are linked; or preferably

HCV particles obtained from a chimeric HCV genome, in which the nucleotide sequences of, in order from the 5′ side to the 3′ side, the 5′ untranslated region, the core protein coding region, the E1 protein coding region, the E2 protein coding region, the p7 protein coding region, and a sequence encoding 16 amino acid residues from the N terminal amino acid residue of the NS2 protein coding region from the J6CF strain, and a portion ranging from (encoding) the 17th amino acid residue from the N-terminus to the C terminal amino acid residue of the NS2 protein coding region, the NS3 protein coding region, the NS4A protein coding region, the NS4B protein coding region, the NS5A protein coding region, the NS5B protein coding region, and the 3′ untranslated region from the JFH1 strain are linked.

Also, an example of the HCV genome in another preferred embodiment of the present invention is an HCV genome to be used for preparing infectious HCV particles that are used in the present invention, which contains at least nucleotide sequences encoding the core protein, the E1 protein, the E2 protein, and the p7 protein, respectively, that are structural proteins of the J6CF strain of genotype 2a.

The above infectious HCV particles can be prepared by synthesizing RNA from a vector in which the cDNA of the above full-length HCV genome RNA has been cloned downstream of a transcriptional promoter (e.g., a vector in which HCV genome DNA has been cloned under the control of T7 promoter), and then introducing the RNA into cells. When a method for preparing RNA in vitro with the use of the nucleic acid as a template, in which HCV cDNA has been cloned under the control of T7 promoter, is employed, RNA can be synthesized using a MEGAscript T7 kit (Ambion), for example. Cells into which RNA is introduced may be cells that enable the formation of HCV particles and examples thereof include cultured cells such as Huh7 cells, HepG2 cells, IMY-N9 cells, HeLa cells, and 293 cells. More preferable examples thereof include liver-derived cultured cells such as Huh7 cells. Further preferable examples thereof include Huh7 cells and cells of a derivative strain of Huh7 (e.g., Huh7.5 cells and Huh7.5.1 cells). Furthermore, examples thereof include Huh7 cells, HepG2 cells, IMY-N9 cells, HeLa cells, or 293 cells in which CD81 gene and/or Claudin1 gene has been expressed. In particular, Huh7 cells or cells of a derivative strain of Huh7 are preferably used. The term “derivative strain” in the present invention refers to a cell line induced from the relevant cells.

Any known method can be used as a method for introducing RNA into cells. Examples of such a method include calcium phosphate coprecipitation, a DEAE dextran method, lipofection, microinjection, and electroporation. Preferable examples thereof include lipofection and electroporation. A further preferable example thereof is electroporation.

The capacity of cells to produce viral particles can be detected using an antibody against an element (e.g., a core protein, an E1 protein, or an E2 protein) composing HCV viral particles that are released into a culture solution. Also, HCV genome RNA contained in HCV viral particles in a culture solution is amplified by an RT-PCR method using specific primers for detection, so that the presence of HCV viral particles can also be detected indirectly.

Whether or not the prepared viral particles are infectious can be evaluated by treating HCV infection-susceptible cells (e.g., Huh7 cells) with the supernatant obtained by culturing cells into which HCV RNA has been introduced in the aforementioned manner, followed by, for example, after 48 hours, immunologically staining the cells with an anti-core antibody to count the number of infected cells. Alternatively, evaluation can be carried out by subjecting the cell extract to electrophoresis on SDS-polyacrylamide gel and detecting core proteins via Western blotting.

In the description, viral particles prepared from a full-length genome derived from the JFH1 strain are referred to as “JFH1-HCV”, viral particles prepared from a J6/JFH1 genome are referred to as “J6/JFH1-HCV,” and viral particles prepared from a TH/JFH1 genome referred to as “TH/JFH1-HCV.” These viral particles are viruses derived from JFH1.

In the present invention, hepatitis C virus (HCV) particles of genotype 2a are purified as necessary, as described below, and then inactivated, so that the resultant can be used as an antigen for the vaccine composition according to the present invention. Preferable hepatitis C virus (HCV) particles of genotype 2a have structural proteins of hepatitis C virus of genotype 2a, particularly, the structural proteins (the core protein, the E1 protein, the E2 protein, and the p7 protein) derived from J6CF strain. The hepatitis C virus (HCV) particles of genotype 2a preferably have the E2 protein of hepatitis C virus of genotype 2a and particularly, the J6CF-strain-derived E2 protein. A preferable example of the polyprotein region consisting of the structural proteins (the core protein, the E1 protein, the E2 protein, and the p7 protein) derived from J6CF strain consists of an amino acid sequence encoded by the nucleotide sequence ranging from the 341^{st} to 2779^{th} amino acid residues on the sequence shown in SEQ ID NO: 2.

Furthermore, the hepatitis C virus (HCV) particles of genotype 2a are preferably viral particles that are produced by expression of an HCV genome containing nucleotide sequences encoding an NS2 protein derived from at least 1 type of hepatitis C virus strain, and NS3 protein, NS4A protein, NS4B protein, NS5A protein, and NS5B protein derived from the JFH1 strain for the reason that they have infectivity and autonomous replication capacity. Such a hepatitis C virus (particles) of genotype 2a may be a chimeric virus (particles). The hepatitis C virus (HCV) particles of genotype 2a are preferably viral particles produced by the expression of a chimeric HCV genome that comprises: nucleotide sequences encoding a 5′ untranslated region derived from any one of hepatitis C virus strains, the structural proteins (the core protein, the E1 protein, the E2 protein, and the p7 protein) derived from J6CF strain, an NS2 protein which is a chimeric protein derived from the J6CF strain and JFH1 strain (a portion ranging from the N-terminal amino acid residue to the 16″ amino acid residue of the NS2 protein is derived from the J6CF strain, and a portion ranging from the 17^{th} amino acid residue from the N terminal acid residue to the C-terminal amino acid residue of the NS2 protein is derived from the JFH1 strain), and non-structural proteins (NS3 protein, NS4A protein, NS4B protein, NS5A protein, and NS5B protein) derived from the JFH1 strain other than NS2; and a nucleotide sequence containing a 3′ untranslated region derived from any one of hepatitis C virus strains.

### (2) Purification of Viral Particles

The virus solution containing the HCV particles obtained in (1) above is subjected to, for example, centrifugation and/or filtration through a filter to remove cells and cell residues. A solution from which residues have been removed can also be concentrated approximately 10- to 100-fold using an ultrafiltration membrane with a molecular weight cut off of 100,000 to 500,000. A solution containing HCV particles from which residues have been removed can be purified via chromatography and density-gradient centrifugation (described later) in arbitrary combinations in any order or alone. Hereafter, representative chromatography or density-gradient centrifugation techniques are described, although the techniques are not limited thereto.

In gel filtration chromatography, a chromatography support comprising a cross-linked polymer of allyl dextran and N,N′-methylenebisacrylamide can be preferably used as the gel matrix, and more preferably using Sephacryl (registered trademark) S-300, S-400, and S-500 chromatography to purify infectious HCV particles.

In ion-exchange chromatography, preferably, Q-Sepharose (registered trademark) can be used as an anion exchange resin. That is, HCV particles can be purified using Q-Sepharose, for example. Moreover, preferably, SP Sepharose (registered trademark) or the like is used as a cation exchange resin to purify HCV particles.

In affinity chromatography, a resin binding a substrate selected from among heparin, sulfated cellulofine, lectin, and various dyes as a ligand can be preferably used as a support to purify HCV particles. Further preferably, HCV particles can be purified with the use of a support binding HiTrap Heparin HP (registered trademark), HiTrap Blue HP (registered trademark), HiTrap Benzamidine FF (registered trademark), sulfated cellulofine, LCA, ConA, RCA-120, and WGA. The most preferable method is purification of HCV particles with the use of sulfated cellulofine as a support. HCV particles can be purified 30-fold or more in terms of the ratio of the HCV RNA copy number to the total protein amount in the solution before and after the purification.

In purification via density-gradient centrifugation, a sugar polymer, such as cesium chloride, sucrose, Nycodenz (registered trademark), Ficoll (registered trademark), or Percoll (registered trademark) can be preferably used as a solute that makes density gradient. Sucrose can be further preferably used. Preferably, water or a buffer, such as phosphate buffer, Tris buffer, acetate buffer, or glycine buffer, can be used as a solvent. The centrifugal force that is employed at the time of purification via density-gradient centrifugation is preferably ranges from 1×10⁴ g to 1×10⁹ g, more preferably ranges from 5×10⁴ g to 1×10⁷ g, and most preferably ranges from 5×10⁴ g to 5×10⁵ g.

Purification is carried out preferably at 0° C. to 40° C., more preferably at 0° C. to 25° C., and most preferably at 0° C. to 10° C.

After concentration of HCV particles with an ultrafilter membrane, HCV particles can also be purified by density-gradient centrifugation. Furthermore, when purification is carried out via density-gradient centrifugation in combination with column chromatography, such techniques may be carried out in any combination in any order. Preferably, HCV particles are first purified through a plurality of chromatography columns followed by density-gradient centrifugation. More preferably, a fraction containing HCV particles obtained via anion exchange column chromatography followed by affinity chromatography is subjected to purification via density-gradient centrifugation. Most preferably, a fraction containing HCV particles obtained with the use of a Q-Sepharose (registered trademark) column is further purified with the use of a sulfated cellulofine-based column, and the resulting fraction containing HCV particles is then purified via density-gradient centrifugation. During the steps of column chromatography and density-gradient centrifugation, dialysis or ultrafiltration may be carried out to substitute a solute of a solution containing HCV particles and/or to concentrate HCV particles.

### (3) In Activation of Viral Particles

The vaccine of the present invention reacts with the inactivated HCV particle as an antigen. With regard to the vaccine of the present invention, the presence or the absence of the infectivity of the HCV particles poses no problem for evaluation using rodents, since HCV does not exhibit infectivity to rodents. However, when the vaccine is inoculated to a human, the use of inactivated HCV particles is required. HCV particles can be inactivated by adding and mixing an inactivator such as formalin, β-propiolactone, or glutardialdehyde in, for example, a virus suspension to allow the inactivator to react with viruses (Appaiahgari et al., Vaccine, 22: 3669-3675, 2004). Further, HCV particles may be irradiated with ultraviolet rays to cause the loss of infectivity of viruses, and viruses can be immediately inactivated. Irradiation with ultraviolet rays is preferred since it realizes virus inactivation with little influence on proteins that constitute viruses. A source of ultraviolet rays used for inactivation can be a commercially available germicidal lamp. In particular, a 15 w germicidal lamp can be used, although the source is not limited thereto. In the present invention, HCV particles that are purified by the method described above are used, and methods of inactivation are not limited by a purified or unpurified state. Preferably, a solution containing infectious HCV particles may be irradiated with ultraviolet rays at 20 mW/cm² at room temperature for at least 5 minutes to inactivate infectious HCV particles.

### (4) Adjuvant

Examples of an adjuvant that can be used herein include aluminium hydroxide (Alum) whose use as an adjuvant for vaccines has already been approved, and double-stranded RNA (dsRNA) and unmethylated CpG-containing oligonucleotide (CpG-ODN) for which clinical trials have been conducted.

Examples of dsRNA include polyI:C, polyICLC, and polyIpolyC12U.

Any immunostimulatory oligonucleotide to be contained in the vaccine of the present invention may be used herein as long as it contains unmethylated CpG. A preferable example thereof is an oligonucleotide disclosed in International Patent Publication WO07/139,190. A further preferable example thereof is GGGGGGGCGACGATCGTCAGG (SEQ ID NO: 4, referred as Mod87).

Degradation of an oligonucleotide having the phosphodiester backbone is mediated by exonuclease and endonuclease. It is known that phosphorothioate modification of nucleotide-to-nucleotide bonds results in acquisition of resistance to these nucleases. Examples of an oligonucleotide in which some bonds between nucleotide residues are modified include oligonucleotides in which 5′ and 3′ terminal nucleotides are linked via phosphorothioate-modified bonds and oligonucleotides in which nucleotides of a polyG sequence are linked via phosphorothioate-modified bonds. These oligonucleotides have resistance to exonuclease.

Therefore, a more preferred example of an oligonucleotide is EEEEEEECGACGATCGTCAEG (SEQ ID NO: 5; phosphorothioated guanine (G) is denoted as “E” and referred to as “Mod87s”), wherein the phosphodiester bond between 5′-terminal polyG sequence and the end on the 3′ terminal side of the oligonucleotide of SEQ ID NO: 4 has undergone phosphorothioate modification.

### (5) Vaccine Composition and its Effects

The hepatitis C virus vaccine composition according to the present invention can be produced by mixing inactivated hepatitis C virus (HCV) particles produced by the above-mentioned method or the like with adjuvants, particularly unmethylated CpG-containing oligonucleotide shown in SEQ ID NO: 5 in the sequence listing and aluminium hydroxide by a conventional method. The vaccine composition of the present invention contains inactivated HCV particles as antigens in an amount ranging from 0.001 to 99.999% by weight.

Also, dosage can be appropriately determined depending on subjects' age, patients' symptoms, therapeutic purposes, routes of administration, and the like. Any dosage may be employed herein, as long as the amount is sufficient for activation of immunity capable of preventing HCV infection or the onset of HCV. In general, dosage preferably ranges from 0.1 mg to 10000 mg, and more preferably ranges from 1 mg to 100 mg per administration to an adult. Also, after initial administration, specifically 2 weeks, 4 weeks, 6 weeks, 8 weeks, 2 months, 3 months, 4 months, 5 months, 6 months, or 1 year or more later, one or more booster doses of the vaccine composition of the present invention are provided. Thus, immune-response-inducing capacity can be maintained. Therefore, the frequency of administration is preferably 2 or more times, furthermore preferably ranges from 2 to 6 times, more preferably ranges from 3 to 5 times, and is most preferably 4 times.

The effects of the vaccine composition of the present invention can be evaluated by administering the vaccine to an animal and then detecting an anti-HCV antibody induced by the immune reaction. Animals to be used for immunization may be any animals, as long as they are non-human animals (e.g., non-human mammals) such as chimpanzees, monkeys, mice, rats, hamsters, and rabbits. In the present invention, such animals are preferably mice. Examples in which mice are used are as described below.

In general, a 4- to 10-week-old mouse is immunized by administering several times the HCV particles obtained by the above steps (1) to (3) as antigens and an adjuvant to the mouse. As such an adjuvant, Alum, CpG-ODN, and dsRNA described in (4) can be used independently or in combination.

After administration of the vaccine composition, blood is collected via tail vein of the mouse to which the vaccine has been administered. The antibody titers against the HCV proteins in serum are measured as described in (6) below, so that the degree of immune response induced by the vaccine composition can be measured. HCV is known to infect cells via the envelope proteins. Hence, it is preferred to measure the antibody titers against envelope proteins (e.g., E1 protein and E2 protein). Further preferably, the activity of inhibiting HCV infection is measured as described in (7) below, so that the effects of the vaccine composition can be examined.

### (6) Measurement of Antibody Titer

For evaluation of the effects of the vaccine composition according to the present invention, an HCV protein is fixed (immobilized) to a support. Next, serum is added, and then an antibody in serum and the immobilized antigen are caused to react for sufficient time under sufficient conditions for the formation of a complex. Subsequently, the thus generated complex is brought into contact with an antibody (secondary antibody) recognizing the antibody (in the serum) bound with an enzyme, a dye, or a radioisotope as a signal, so that a 2nd mixture is generated. The 2nd mixture is caused to react for sufficient time under sufficient conditions for the formation of an antibody-antigen complex. The presence of the antibody recognizing the HCV protein is detected by detecting the signal from the enzyme, dye, or radioisotope.

As such an HCV protein to be immobilized to a support, an HCV particle can be directly used. Alternatively, cDNA consisting of the nucleotide sequences of, in the HCV genome, a core protein coding region, an E1 protein coding region, an E2 protein coding region, a p7 protein coding region, an NS2 protein coding region, an NS3 protein coding region, an NS4A protein coding region, an NS4B protein coding region, an NS5A protein coding region, and/or an NS5B protein coding region can be used. Proteins encoded by these regions expressed in Escherichia coli, yeast, mammalian cells, insect cells, or the like can also be used. Furthermore, such proteins can be chemically synthesized and then used.

Methods for expressing the envelope proteins of the J6CF strain in mammalian cells are as described in JP Patent Application No. 2007-193413 and JP Patent Application No. 2008-254338. Specifically, when the initiator methionine of the amino acid sequence (NCBI Protein Accession No. AAF01178.1) of a full-length protein of the J6CF strain is regarded as No. 1, the E1 protein of the J6CF strain starts from the 192nd amino acid residue and ends at the 383rd amino acid residue. The E2 protein of J6CF strain starts from the 384th amino acid residue and ends at the 750th amino acid residue of the above amino acid sequence. The portion ranging from the 353rd to the 383rd amino acid residues of the E1 protein and the portion ranging from the 722nd to the 750th amino acid residues of the E2 protein are thought to be transmembrane domains (Cocquerel, L. et al., J. Virol. 74: 3623-3633, 2000).

For secretory expression of proteins in a culture supernatant by mammalian cells, the proteins are required to have a signal peptide but no transmembrane domain.

Therefore, in the case of the E1 protein of the J6CF strain, a sequence ranging from the 192nd to the 352nd amino acid residues can be used as such a protein having no transmembrane domain. In the case of the E2 protein of the same, a sequence ranging from the 384^{th} to the 720th amino acid residues can be used as such a protein having no transmembrane domain.

For secretory expression of the E1 or E2 protein having no transmembrane domain by mammalian cells, a nucleic acid encoding the protein is ligated downstream of a nucleic acid encoding a signal peptide, such that the reading frames (readable frames) for codons match. Furthermore, a stop codon is added to the 3′ end, and then the resultant is inserted to an expression vector. A signal peptide consists mainly of hydrophobic amino acids; that is, 15 to 30 amino acid residues existing on the N terminus of a secretory protein, and is involved in the mechanisms of protein transport through the cell membrane

A signal peptide that can be used for secretory expression of a protein by mammalian cells may be of any secretory protein. Examples of a vector having a signal peptide include a vector having the signal peptide sequence of mouse GM-CSF (JP Patent Publication (Kokai) No. 63-276490 A (1988)), a pSecTag/FRT/V5-His vector (Invitrogen) having the signal peptide sequence of IgG κ chain, a p3×FLAG-CMV13 vector (Sigma) having the signal peptide sequence of preprotrypsin, a pFUSE-Fc2 vector (InvivoGen) having the signal peptide sequence of IL-2, and a pTriEx-7 vector (Novagen) having the signal peptide sequence of IgM.

When proteins are expressed, such proteins are expressed as fusion proteins of target proteins and label proteins, and fusion proteins can be detected or purified with the use of an antibody reacting with the label protein or a molecule that specifically binds thereto. Such label proteins are also referred to as “tags.” Label proteins are not limited, and examples thereof include FLAG peptide (also referred to as flag peptide or Flag peptide), 3× FLAG peptide (also referred to as 3× FLAG peptide, 3× Flag peptide, or 3× flag peptide), HA peptide, 3× HA peptide, myc peptide, 6× His peptide, GST polypeptide, MBP polypeptide, PDZ domain polypeptide, alkaline phosphatase, immunoglobulin, and avidin. Such peptides or polypeptides are generally fused to the N- or C-terminus of the target proteins, but such peptides or polypeptides can be inserted into the target proteins according to need. A vector having a fusion polypeptide of a preprotrypsin signal peptide and a 3× FLAG peptide is available as the p3×FLAG-CMV-9 vector from Sigma.

### (7) Measurement of Inhibition of Infection

To measure whether or not an animal serum sample obtained after administration of the vaccine composition has the activity of inhibiting HCV infection, infectious HCV-like particles (HCVpp) or infectious HCV particles (HCVcc) can be used.

Infectious HCV-like particles can be prepared by causing retrovirus particles to present functional HCV envelope proteins thereon. A green fluorescent protein marker gene or a luciferase gene is packaged within these infectious HCV-like particles, making it possible to rapidly measure with high reliability infection mediated by HCV envelope proteins (Bartosch, B. et al. J. Exp. Med. 197: 633-642, 2003).

Specifically, for example, a pcDNA J6dC-E2 vector is constructed by cloning a nucleic acid that encodes the 132nd to the 750th amino acid residues (a part of the core protein, the E1 protein, and the E2 protein) of the protein of J6CF (NCBI Protein Accession No. AAF01178.1) which is an HCV strain of genotype 2a into pcDNA3.1. A Gag-Pol 5349 expression vector is constructed by cloning genes that encodes gag and pol of a mouse leukemia virus into the vector. A Luc126 retrovirus vector is constructed by cloning a luciferase gene into the vector. The vectors are transfected into 293T human embryo renal cells (ATCC CRL-1573) using FuGENE6, so that HCVpp can be produced. After transfection, a culture solution containing pseudo particles is collected and then filtered with a 0.45-μm membrane filter, so that it can be used as an HCVpp sample.

For preparation of HCVpp having envelope proteins of genotype 1a, a pcDNA H77dC-E2 vector constructed by cloning a nucleic acid that encodes the 132nd to the 746th amino acid residues (a part of the core protein, the E1 protein, and the E2 protein) of the protein (NCBI Protein accession No. AAB67036.1) of H77 (HCV of genotype 1a) into pcDNA3.1 can be used. For preparation of HCVpp having envelope proteins of genotype 1b, a pcDNA THdC-E2 vector constructed by cloning a nucleic acid encoding the 132nd to the 747th amino acid residues (a part of the core protein, the E1 protein, and the E2 protein) of a protein of TH (HCV of genotype 1b) (Wakita, T. et al., J. Biol. Chem., 269, 14205-14210, 1994) into pcDNA3.1 can be used.

For example, HCVpp is mixed with a diluted serum sample, and then the mixture is allowed to react at room temperature for 30 minutes. Serum is diluted with a DMEM medium (DMEM containing 10% FCS (Invitrogen), a 1% MEM nonessential amino acid solution (Invitrogen), 10 mM HEPES-Tris (pH 7.3), and 1 mM sodium pyruvate). The above mixture of HCVpp and the serum sample is added to Huh7.5.1 cells cultured on the previous day in 96-well plate at 1×10⁴ cells/well, followed by 3 hours of culture at 37° C. After culture, the sample is removed, cells are washed once with PBS, a fresh medium is added, and then cells are cultured continuously. After 72 hours, a culture supernatant is removed, washing is performed 4 times with PBS, 25 μL/well DMEM medium and 25 μL/well Steady-Glo (Promega: catalog No. E2520) are added, and then cells are lysed according to instructions included therewith. The cell lysis solution (40 μL/well) is transferred to a white 96-well plate (Sumitomo Bakelite Co., Ltd.: catalog No. MS-8496W), and then luminescence intensity is measured using ARVO X4 (PerkinElmer).

A value obtained after mixing with DMEM is regarded as 100% infection. Luciferase activity after mixing with a serum sample is expressed with percentages (%) and thus the activity of inhibiting HCV infection (%) can be found.

Also, for example, HCVcc is mixed with a diluted serum sample, and then the mixture is allowed to react at room temperature for 30 minutes. Serum is diluted with a DMEM medium (DMEM containing 10% FCS (Invitrogen), a 1% MEM nonessential amino acid solution (Invitrogen), 10 mM HEPES-Tris (pH 7.3), and 1 mM sodium pyruvate). The mixture of HCVcc such as J6/JFH1-HCV particles and the serum sample is added to Huh7.5.1 cells cultured on the previous day in a 96-well plate at 1×10⁴ cells/well, followed by 3 hours of culture at 37° C. After culture, the sample is removed, cells are washed once with PBS, a fresh medium is added, and then cells are cultured continuously. After 72 hours, a culture supernatant is removed and then the plate is soaked in ice-cooled methanol, so as to immobilize cells. Subsequently, methanol is removed by air drying and then cells are solubilized using BlockAce (registered trademark) (Dainippon Pharmaceutical) containing 0.3% Triton (registered trademark)-X 100 (GE Healthcare). An HRP-labeled anti-HCV-core antibody (Ortho Clinical Diagnostics) is reacted, and then hepatitis C virus-infected cells are detected using a QuantaBlu (registered trademark) (PIERCE) reaction solution. After addition of a QuantaBlu stop solution, 20 μL/well of the resultant is transferred to a black 384-well plate (Corning: catalog No. 3676), and then fluorescence intensity is measured using ARVO X4 (PerkinElmer).

The value obtained after mixing with DMEM is regarded as representing 100% infection. QuantaBlu fluorescence intensity after mixing with a serum sample is expressed with percentages (%), and thus the activity of inhibiting HCV infection (%) can be found.

As confirmed above, the vaccine composition according to the present invention has significantly strong activity of inhibiting HCV infection. Moreover, the vaccine composition is advantageous in that it can exhibit strong activity of inhibiting HCV infection not only for HCV of genotype 2a used as an antigen, but also for HCVs of other types of genotype (e.g., 1a, 1b, and 2b).

### EXAMPLES

The present invention will be described in more detail with reference to the examples set forth below.

### Example 1

### Preparation of J6/JFH1-HCV Particles

cDNA (genomic cDNA) was obtained by reverse transcription of the full genomic RNA region of the hepatitis C virus (HCV) JFH1 strain (genotype 2a) separated from a fulminant hepatitis patient. The cDNA was cloned downstream of the T7 RNA promoter sequence of a pUC19 plasmid, so as to obtain a pJFH1 plasmid DNA (Wakita, T. et al., Nat. Med., 11 (2005) p. 791-796 and International Patent Publication WO 2004/104198). The pJFH1 plasmid DNA was digested with EcoR I and then partially digested with Bcl I. A fragment (about 2840 bp) ranging from the EcoR I site to the first Bcl I site was excised so as to prepare a plasmid DNA fragment, and then the plasmid DNA fragment was purified.

Meanwhile, HCV J6CF-strain-derived genomic cDNA (GenBank Accession No. AF177036, Yanagi, M., et al., Virology 262: 250-263 (1999)) was cloned downstream of the T7 RNA promoter sequence of a pUC19 plasmid, so as to obtain a pJ6CF plasmid DNA (International Patent Publication WO 2006/022422). The pJ6CF plasmid DNA was partially digested with EcoR I and Bcl I, and then the thus obtained about 2840-bp fragment was purified. The fragment was ligated to the pJFH1 plasmid fragment obtained by excision of the EcoR I-Bcl I fragment, so that a pJ6/JFH1 plasmid DNA was prepared. The DNA (SEQ ID NO: 2) cloned into pJ6/JFH1 is the genomic cDNA of a chimera virus prepared by ligating a sequence encoding the region ranging from the 17th amino acid residue to the C terminal amino acid residue of the NS2 protein, sequences encoding NS3, NS4A, NS4B, NS5A, and NS5B proteins in this order, and the 3′ untranslated region of the genomic cDNA of the JFH1 strain to the 5′ untranslated region, sequences encoding core, E1, E2, and p7 proteins, and the sequence encoding the region ranging from the N terminal amino acid residue to the 16th amino acid residue of the NS2 protein in the genomic cDNA of the J6CF strain.

After cleavage of the pJ6/JFH1 with Xba I, Mung Bean Nuclease 20U (total reaction solution volume: 50 μL) was added followed by 30 minutes of incubation at 30° C., so as to blunt-end the terminus cleaved with Xba I. Subsequently, phenol.chloroform extraction and ethanol precipitation were performed, so that a fragment cleaved with Xba I, from which 4 bases (CTAG) had been removed, was obtained. RNA was synthesized using the DNA as a template and a MEGA script T7 kit (Ambion). The thus synthesized HCV RNA was used for the following introduction into cells.

3×10⁶ Huh-7 cells and 5 μg of HCV RNA were suspended in a Cytomix solution (120 mM KCl, 0.15 mM CaCl₂, 10 mM K₂HPO₄/KH₂PO₄, 25 mM Hepes, 2 mM EGTA, 5 mM MgCl₂, 20 mM ATP, 50 mM glutathione 400 μL). After the suspension was transferred to a 4-mm cuvette, HCV RNA was electroporated into Huh-7 cells using a Gene Pulser (BioRad) at 260 V and 950 μF. Subsequently, cells into which the HCV RNA had been introduced were seeded on a 10-cm² dish, and then subcultured. Upon subculture, the HCV core protein contained in culture supernatants was quantified using the HCV antigen ELISA test kit (Ortho Clinical Diagnostics) to confirm the production of HCV particles. Culture supernatants containing the core protein at high levels and exhibiting high activity of producing HCV particles were selected and then stored as virus stocks.

About 100 μL each of the above obtained J6/JFH1 virus stock (4×10⁴ ffu/mL) was added to Huh-7 cells cultured with 10% FCS-DMEM medium (1% MEM nonessential amino acid solution (Invitrogen), 10 mM HEPES-Tris (pH 7.3), 1 mM sodium pyruvate) in the 10-cm dish, so that Huh-7 cells were infected with the HCV virus.

The cells were adequately subcultured while avoiding cells from becoming confluent, and then culture expansion was performed from one to six 225-cm² flasks. Subsequently, cells were detached from five 225-cm² flasks, seeded in three 5-layer Cellstacks (registered trademark) (Corning), a medium was added thereto in an amount of 650 mL/cell stack. The cells obtained from the other 1 flask were seeded in 6 flasks and thus virus production could be efficiently continued.

On the day following subculture, the media were discarded and 650 mL of 2% FCS-DMEM medium (1% MEM nonessential amino acid solution (Invitrogen), 10 mM HEPES (pH 7.3), 1 mM sodium pyruvate) was added. The media were recovered 3 days after medium exchange, and then caused to pass through a 0.45-μm filter. The resultants were stored in a deep freezer. Also, 650 mL of 2% FCS-DMEM medium (1% MEM non-essential amino acid solution, 10 mM HEPES (pH 7.3), and 1 mM sodium pyruvate) was added to the Cellstacks after the culture supernatants had been recovered, and culture was continued. A similar procedure was repeated 2 days after the medium exchange and the culture supernatants were recovered. A similar procedure was then repeated one more time. The culture supernatants recovered herein were used as culture supernatants containing J6/JFH1-HCV particles in the Examples below.

### Example 2

### Purification of J6/JFH1-HCV Particles

J6/JFH1-HCV particles produced in Example 1 were purified via the following 3 steps.

### 1) Concentration and Diafiltration

The culture supernatants containing HCV particles obtained in Example 1 were concentrated 60-fold using a Hollow Fiber Cartridge (GE Healthcare: 500 kDa cut-off model No. UFP-500-C-8A, hereinafter, referred to as “Hollow Fiber”).

### 2) Density-Gradient Ultracentrifugation

To the Ultra-clear 25 mm×89 mm centrifuge tube (Catalog No. 344058, Beckman Coulter), 3 mL of TNE buffer containing cold 60% sucrose (10 mM Tris-HCl (pH 7.4), 150 mM NaCl, 1 mM EDTA) was added, and 7 mL of TNE buffer containing 20% sucrose was overlaid thereon. Further, 25 mL of the sample was overlaid onto the TNE buffer containing 20% sucrose. Ultracentrifugation was carried out using a SW-28 (Beckman Coulter) at 28,000 rpm for 4 hours at 4° C.

The bottom of the tube was perforated using the 25 G injection needle (Terumo) and twelve 1-mL fractions were obtained. Specific gravity was measured for the solution of each fraction, so that the formation of the density gradient of sucrose was confirmed. Fractions with 3rd, 4th, and 5th largest specific gravity were recovered in descending order and then used for concentration and buffer exchange.

### 3) Concentration and Buffer Exchange

The elution fraction was subjected to buffer exchange and concentration using Amicon Ultra-15 Centrifugal Filter Units (molecular weight to be excluded: 100 kDa, Millipore) and TNE buffer. The thus-obtained concentrate was used as a virus solution containing infectious HCV particles in the immunization step described below.

### Example 3

### Inactivation of HCV Particles

The concentrated hepatitis C viruses obtained by steps 1) to 3) in Example 2 were inactivated via ultraviolet irradiation. As a source of ultraviolet rays, a GL-15 (Toshiba) was used. Specifically, the solution containing purified hepatitis C virus particles (the JFH-1 strain) having an infectious titer of 1×10⁶ ffu/mL was introduced into a silicon-coated polyethylene Eppendorf tube (Assist Co., Ltd), the tube was placed at a distance from the source of ultraviolet rays, so that the ultraviolet rays would be applied at the intensity of 20 mW/cm², and UV-C was applied for 5 minutes.

After ultraviolet irradiation, hepatitis C virus particles were serially diluted 50-fold, 250-fold, 1,250-fold, 6,250-fold, 31,250-fold, 156,250-fold, and 781,250-fold in Dulbecco's modified Eagle medium (DMEM).

On the previous day, the Huh-7 cells were seeded on a 96-well poly-L-lysine-coated plate (Corning 96 Well Clear Flat Bottom Poly-L-Lysine Coated Microplate) at 1×10⁴ cells/well, the serially-diluted viral particles were seeded thereonto, and culture was conducted at 37° C. for 72 hours.

After the culture supernatant was removed, the plate was soaked in ice-cold methanol to fix the cells. Thereafter, methanol was removed via air drying, and cells were solubilized with the use of Block Ace (registered trademark) (Dainippon Pharmaceutical Co., Ltd.) containing 0.3% Triton (registered trademark)-X 100 (GE Healthcare). HCV-infected cells were detected using the clone 2H9 anti-HCV-core antibody (Wakita, T. et al., Nat. Med. 11: 791-796, 2005) and goat anti-mouse IgG-Alexa488 (Molecular Probes), and the HCV-infected cells were counted under a fluorescent microscope (IX-70; Olympus). The infectious titer of the ultraviolet-irradiated hepatitis C viruses was found to be the same or below the detection limit. HCV particles that had been caused to completely lose infectivity were administered to mice in the Examples below.

### Example 4

### Preparation of J6E1Fc Protein and J6E2Fc Protein

The J6E1Fc protein and the J6E2Fc protein were prepared by the following method.

(1) Construction of Vector for Expression of Fusion Protein of E1 Protein Derived from J6CF Strain of HCV2a Added to Fc Protein of Human IgG

A gene encoding the E1 protein consisting of a region ranging from the 192nd to the 353rd amino acid residues of the precursor protein of the J6CF strain, when the initiation methionine at the N-terminus was designated as the 1st amino acid residue, was amplified by a PCR method using the cDNA of the genomic RNA of the J6CF strain of HCV2a (GenBank Accession No. AF177036) as a template, an Advantage GC1 PCR kit (Takara Bio Inc.), and J6E1F-s (SEQ ID NO: 9: CACAAGCTTGCCGAAGTGAAGAACATCAGT) and J6E1F-as (SEQ ID NO: 10: TCGGGATCCCAATGAGCCCCGCTAATGATGTC) as primers.

Next, the fragment amplified by the PCR method was cloned into pCR-TOPO (Invitrogen) and then 3 clones were subjected to sequence analysis. Clones with correct nucleotide sequences were designated as “pTOPO-J6E1F.”

Next, p3×FLAG-CMV-13 (Sigma) was digested with Hind III and BamH I, separated by agarose gel electrophoresis, and then purified. The thus obtained DNA fragment and a DNA fragment containing cDNA encoding the E1 protein of J6CF (obtained by digestion of pTOPO-J6E1F with Hind III and BamH I) were cyclized with T4 DNA ligase. The vector was designated as “CMV-13-J6E1F.” CMV-13-J6E1F was digested with Sac I and BamH I, the DNA fragment encoding the E1 protein of JFH1 was separated by agarose gel electrophoresis and then purified using GeneElute (Sigma).

A CDM-mIL7R-Ig vector (Sudo et al., Proc Natl. Acad Sci U.S.A., 1993, Vol. 90, p. 9125-9129) expressing a chimeric protein comprising a mouse IL-7 receptor-human immunoglobulin Fc domain was digested with Sac I and BamH I. A DNA fragment containing the sequence encoding a human immunoglobulin Fc region was separated by agarose electrophoresis and then purified. The DNA fragment and the DNA fragment encoding the above J6 E1 protein were linked with T4 DNA ligase. Thus, a “CDM-J6E1Fc” vector expressing the chimeric protein (hereinafter, referred to as “J6E1Fc protein”) consisting of the J6E1 protein and the immunoglobulin Fc domain was obtained.

(2) Construction of Vector for Expression of Fusion Protein of E2 Protein Derived from the J6Cf Strain of HCV2a Added to Fc Protein of human IgG

First, a gene encoding a protein consisting of a region corresponding to the 384th to 720th amino acid residues of the precursor protein of the J6CF strain, when the initiation methionine at the N-terminus was designated as the 1st amino acid, was amplified by a PCR method using the cDNA (GenBank Accession No. AF177036) of the genomic RNA of the J6CF strain of HCV2a as a template, an Advantage GC2 PCR kit (Takara Bio Inc.), and J6E2Fc-s (SEQ ID NO: 6: CACAAGCTTCGCACCCATACTGTTGGGG) and J6E2Fc-as (SEQ ID NO: 7: ACAGGATCCCATCGGACGATGTATTTTGTG) as primers.

Next, the thus amplified DNA fragment was cloned into pCR-TOPO (Invitrogen) and then 3 clones were subjected to sequence analysis. A gene fragment encoding the antigen E2 protein was digested with Hind III and BamH I and thus excised from clones having the correct nucleotide sequence insert and then inserted in-frame between the Hind III site and the BamH I site downstream of a signal peptide sequence of p3×FLAG-CMV-13 (Sigma). The vector was designated as CMV-13-J6E2.

Subsequently, CMV-13-J6E2 was digested with Sac I and BamH I. The thus generated DNA fragments encoding the signal peptide sequence and the antigen E2 protein, respectively, were each separated by agarose gel electrophoresis, and then purified using GeneElute (Sigma).

Thereafter, DNA fragments encoding the above signal peptide sequence and the antigen E2 protein, respectively, were inserted in-frame between the Sac I site and the BamH I site of the CDM-mIL7R-Ig vector (Sudo et al., Proc Natl. Acad Sci U.S.A., 1993, Vol. 90, p. 9125-9129) expressing the chimeric protein comprising mouse IL-7 receptor-human immunoglobulin Fc domain. Thus a CDM-J6E2Fc vector expressing the antigen E2 protein to which the human immunoglobulin Fc domain had been added (hereinafter, J6E2Fc protein) was obtained.

(3) Expression of Fusion Protein of HCVE1 Protein or HCVE2 Protein with IgGFc Protein

CDM-J6E1Fc or CDM-J6E2Fc was introduced into COS1 cells derived from monkey kidney and then each fusion protein was expressed as described below.

First, COS1 cells were subcultured in RPMI1640 medium (Invitrogen) containing 10% fetal calf serum (Invitrogen), 100 U/mL penicillin, and 100 μg/mL streptomycin. On the day before the gene transfer, COS1 cells were seeded in 150 cm² culture flasks (Corning Coaster Corporation) at a dilution ratio of 1:2 and then cultured overnight at 37° C. in a 5% CO₂ incubator.

Subsequently, DEAE dextran (GE Healthcare) and chloroquine (Sigma) were added to RPMI1640 medium at final concentrations of 400 μg/mL and 100 μM, respectively. 50 μg of the above expression vector (CDM-J6E1Fc or CDM-J6E2Fc) was added at a concentration of 0.1 μg/μL to 13 mL of the solution and then cells were cultured for 3 to 4 days.

After culture, the supernatant of COS1 cells was aspirated off. 10 mL of PBS(−) (Nissui Pharmaceutical Co., Ltd.) was added, and again, PBS(−) was aspirated off for washing cells. Subsequently, a DEAE dextran-DNA mixture was added at 13 mL/150 cm² flask and then the resultant was left to stand at 37° C. in the presence of 5% CO₂.

Four hours later, the DEAE dextran-DNA mixture was aspirated off, each flask was washed once with 10 mL of PBS, Hybridoma-SFM medium (Invitrogen) was added at 50 mL/flask, and then cells were cultured at 37° C. in the presence of 5% CO₂ for 4 days. Thereafter, the culture supernatant was collected in a 50-mL centrifuge tube (Corning Coaster Corporation) and then centrifuged at 2500 rpm for 30 minutes at 4° C. The supernatant was filtered through a 0.2-μm filter (Whatman).

(4) Purification of Fusion Protein of HCVE1 Protein or HCVE2 Protein with IgGFc Protein

The culture supernatant of COS1 cells into which CDM-J6E1Fc or CDM-J6E2Fc had been introduced was subjected to purification using Prosep-A (Millipore) as a carrier to which Protein-A had been bound, as described below.

First, an Econocolumn was filled with 1 mL of Prosep-A, 500 mL of the culture supernatant was caused to pass through at a flow rate of 1-1.5 mL/min, and then washed with 20 mL of PBS(−).

Next, 5 fractions were eluted with 0.1 M Glycine-HCl (pH 3.0) to 1 mL/fraction. Immediately after elution, 1 M Tris-HCl (pH 9.5) was added to return the pH to neutral. 20 μL of each fraction was fractionated under reductive conditions by SDS-polyacrylamide gel electrophoresis, and then stained with Coomassie brilliant blue. As a result, the fusion protein (J6E1Fc protein) consisting of the HCVE1 protein and the human immunoglobulin Fc domain or the fusion protein (J6E2Fc protein) consisting of the HCVE2 protein and the human immunoglobulin Fc domain was purified. The molecular weights under reductive conditions were revealed to be about 60 kDa and about 97 kDa, respectively.

### Example 5

### Preparation of Vaccine Composition Containing Antigen and Adjuvant

A vaccine composition was prepared as an emulsion as described below.

Alum, unmethylated CpG-ODN, and polyI:C were used as adjuvants. As Alum, Imject Alum (registered trademark) (PIERCE: catalog No. 77161) was used. As unmethylated CpG-ODN, Mod87s (SEQ ID NO: 5) was used. polyI:C was purchased from Yamasa Shoyu. These adjuvants were used independently or in combination.

When the adjuvants were used independently, Alum was prepared at 200 μg/100 Mod87s was prepared at 25 μg/100 μL, and polyI:C was prepared at 100 μg/100 μL. Also, when the adjuvants were used in combination (Alum+Mod87s, Alum+poly I:C, or Mod87s+poly I:C), Alum was prepared at 200 μg/50 μL, Mod87s was prepared at 25 μg/50 μL, and polyI:C was prepared at 100 μg/50 and thus two of the three adjuvants were combined and used.

When the J6E2Fc protein described in Example 4 was used as an antigen, 50 μl, of Alum and Mod87s were added to 100 μL of 0.2 μg/μL J6E2Fc protein solution (containing 20 μg of the J6E2Fc protein), so that an emulsion was formed. In addition, when 5 μg of the J6E2Fc protein was used as an antigen, 75 μL of PBS was added to 25 μl of a 0.2 μg/μL J6E2Fc protein solution to 100 μl and then similarly mixed with an adjuvant(s).

When an adjuvant was used independently for 100 μL of a solution containing inactivated J6/JFH1-HCV particles (in an amount equivalent to 2 pmol of HCV core) described in Example 3 to be used as an antigen, Alum, Mod87s, or poly I:C in an amount equivalent thereto was added, so as to form an emulsion. Also, when the adjuvants were used in combination, two adjuvants from among 50 μL of Alum, 50 μL of Mod87s, and 50 μl of poly I:C were combined (a total of 100 μl) and then the combination was added to 100 μl of a solution containing inactivated J6/JFH1-HCV particles, so as to form an emulsion.

### Example 6

### Evaluation of the Immune-Response-Inducing Capacity of Vaccine Composition

### 1. Administration of Vaccine Composition

### 1) Administration of J6E2Fc Chimeric Protein

Balb/c mice (5-week-old, female) were immunized via intraperitoneal administration of an emulsion containing the J6E2Fc protein (5 μg or 20 μg) prepared in Example 5. Two weeks later, a similarly prepared emulsion containing the J6E2Fc protein was similarly administered intraperitoneally for further immunization. Serum samples were prepared from blood collected on day 10 after the final immunization.

In addition, saline was administered as a control.

2) Administration of Inactivated J6/JFH1-HCV particles

Balb/c mice (5-week-old, female) were immunized via intraperitoneal administration of an emulsion containing inactivated J6/JFH1-HCV particles (in an amount equivalent to 2 pmol of HCV core) prepared in Example 5. Two weeks later, a similarly prepared emulsion containing inactivated J6/JFH1-HCV particles was similarly administered intraperitoneally for further immunization. Furthermore, 4 weeks later and 6 weeks later, intraperitoneal administration was similarly performed for immunization. Serum samples were prepared from blood collected 1 week after the final immunization (7 weeks after the primary immunization).

In addition, saline was administered as a control.

### 2. Measurement of Antibody Titers E1 Protein and E2 Protein

Antibody titers against the E1 protein and the E2 protein in the serum samples of mice to which each vaccine composition had been administered as described in “1. Administration of vaccine composition” above were measured. Antibody titers were measured by immobilizing the E1 protein or the E2 protein on a plate and determining by EIA whether antibodies in the serum of each mouse inoculated with each vaccine bound to the E1 protein or the E2 protein immobilized on the plate.

### 1) Preparation of J6CF Strain-E2 Protein

The E2 protein of the J6CF strain was prepared as follows. The gene encoding the E2 protein having no transmembrane region and corresponding to the 384th to the 720th amino acid residues when the N-terminal initiation methionine of the J6CF full-length protein sequence (the protein sequence encoded by the genome sequence of the J6CF strain; the amino acid sequence under GenBank Accession No. AF177036) was regarded as the 1st amino acid residue was amplified by a PCR method using genomic cDNA derived from the J6CF strain of genotype 2a (GenBank Accession No. AF177036) as a template, an Advantage GC2 PCR kit (Takara Bio Inc.), and J6E2Fc-s (SEQ ID NO: 6: CACAAGCTTCGCACCCATACTGTTGGGG) and J6E2dTM-as (SEQ ID NO: 8: GCTCTAGATTACCATCGGACGATGTATTTTGT). The amplified DNA fragment was cloned into pCR-TOPO (Invitrogen) and then 3 clones were subjected to nucleotide sequence analysis. Clones with correct nucleotide sequence inserts were designated as “pTOPO-J6E2dTM.”

Subsequently, DNA obtained by digesting p3×FLAG-CMV-9 (SIGMA) with Hind III and Xba I was ligated to the DNA fragment of approximately 1,000 bp excised from pTOPO-J6E2dTM with Hind III and Xba I for cyclization with the aid of T4 DNA ligase. The resulting vector was designated as CMV-3×FLAGJ6E2dTM.

CMV-3×FLAGJ6E2dTM was introduced into COS1 cells derived from the monkey kidney (Accession Number RCB0143, obtained from Riken Cell Bank) to express proteins therein as described below.

The COS1 cells were cultured in Dulbecco's MEM (D-MEM: Invitrogen) containing 10% fetal calf serum (Invitrogen), 100 U/mL penicillin, and 100 μg/mL streptomycin sulfate. The COS1 cells were seeded in a 150-cm² culture flask (Corning Coaster) at a dilution ratio of 1:2 on the previous day of gene transfer, and the cells were cultured at 37° C. in a 5% CO₂ incubator overnight.

Separately, DEAE dextran (Pharmacia) and chloroquine (SIGMA) were added to D-MEM medium (Invitrogen) to result in final concentrations of 400 μg/mL and 100 μM, respectively. Furthermore, 50 μg of the CMV-3×FLAGJ6E2dTM expression vector was added at a concentration of 0.1 μg/μL to 13 mL of the solution, and culture was then performed. Subsequently, the supernatant of the cultured COS1 cells was aspirated off, and 10 mL of PBS (−) (Nissui) was added thereto and the cells were washed once. After PBS(−) was aspirated off, 13 mL of the DEAE dextran-DNA mixture was added to each 150-cm² flask, and incubation was then carried out at 37° C. in the presence of 5% CO₂ for 4 hours.

Four hours later, the DEAE dextran-DNA mixture was aspirated off, and the cells were washed once with 10 mL of PBS. CHO-SFM medium (Invitrogen) was added thereto in amounts of 50 mL per flask, and culture was performed at 37° C. in the presence of 5% CO₂. The culture supernatant was collected in a 50-mL centrifuge tube (Corning Coaster) 4 days later. The collected supernatant was centrifuged at 6,000 rpm (with the use of a HITACHI RPR9-2 rotor) for 30 minutes at 4° C. and filtered through a 0.2-μm filter (Whatman).

The filtered culture supernatant was purified with the use of anti-FLAG M2 agarose (SIGMA) as follows. To 500 mL of the culture supernatant, 1 mL of anti-FLAG M2 agarose was added, and the reaction was allowed to proceed at 4° C. (in a low-temperature chamber) for 2 hours while undergoing agitation in a spinner bottle. A mixture of the supernatant and anti-FLAG M2 agarose was transferred to the Econo-Column (BIO-RAD) 2 hours later, and flow-through fractions were collected. Subsequently, the column was washed twice with 10 mL of TBS (50 mM Tris-HCl, 150 mM NaCl, pH 7.4). Six fractions (1 mL of each fraction) were eluted with the use of 0.1M glycine-HCl (pH 3.5). Immediately after elution, 1M Tris-HCl (pH 9.5) was added for neutralization. The fractions (20 μl each) were subjected to SDS-polyacrylamide gel electrophoresis under reductive conditions and stained with Coomassie brilliant blue. As a result, E2 proteins derived from the J6CF strain were confirmed to be purified. The protein is designated as “J6E2dTM protein.”

### 2) Measurement of Antibody Titer Against Envelope Protein in Serum Sample

Serum obtained via blood collection from mice to which each vaccine composition was administered in “1. Administration of vaccine composition” above were subjected to measurement of antibody titers against the E1 protein and the E2 protein of the J6CF strain by the following method. The J6E1Fc protein was used as an antigen for antibody titer against the J6CF-strain-derived E1 protein. The J6E2Fc protein or the J6E2dTM protein was used as an antigen for antibody titer against the J6CF-strain-derived E2 protein. After dilution of such an antigen with PBS to 1 μg/mL, the solution was added at 50 μL/well to an immunoplate (Nunc: catalog No. 439454), and then incubated at room temperature for 2 hours or overnight at 4° C. for immobilization. The antigen solution was discarded, Blocking One (NACALAI TESQUE, INC.: catalog No. 03953-95) diluted 5-fold with MilliQ water was added at 200 μL/well, and then incubation was performed at room temperature for 1 hour or overnight at 4° C., so that blocking was performed. After the completion of blocking, washing was performed twice with 0.05% Tween20/PBS (150 μL/well), and then serum diluted 1000- to 10000-fold with 0.05% Tween20/PBS was added at 50 μL/well, followed by 1.5 hours of reaction at room temperature. After completion of the reaction, the resultant was washed 3 times with 0.05% Tween20/PBS 150 μL/well, and then horseradish peroxidase conjugated anti-mouse IgG (GE Healthcare) diluted 5000 fold with 0.05% Tween20/PBS was added at 50 μL/well, followed by 1 hour of reaction at room temperature. After completion of the reaction, the resultant was washed 4 times with 0.05% Tween20/PBS, a staining solution (containing a substrate solution in an amount 1/100 of the solution) of a peroxidase staining kit (Sumitomo Bakelite Co., Ltd.: catalog No. ML-1120T) was added at 50 μL/well, and then incubation was performed at room temperature for 5 minutes to 15 minutes. A stop solution was added at 50 μL/well to stop the reaction, and then absorbance at 450 nm was measured using Benchmark Plus (Bio-Rad).

### 3. Measurement of Neutralization Titer

Effects of vaccines were examined by determining whether or not antibodies in serum samples obtained via blood collection from mice to which each vaccine composition of “1” above had been administered were able to inhibit infection with HCVpp or HCVcc.

### 1) Preparation of Infectious HCV-Like Particles (HCVpp)

HCVpp was prepared according to the method of Bartosch et al (document: Bartosch, B. et al. (2003) J. Exp. Med., 197, 633-642). This is a method for preparing a pseudo virus expressing HCV envelope proteins on its surface by which 3 types of vector (a vector expressing retrovirus Gag-pol, a vector expressing HCV envelope proteins, and a retrovirus packaging vector expressing a reporter gene) are expressed in animal cells, so that the reporter gene is packaged.

For preparation of HCVpp having envelope proteins of genotype 2a, a nucleic acid encoding the 132^{nd} to 750^{th} amino acid residues (a part of the core protein, the E1 protein, and the E2 protein) of the protein of the J6CF strain of HCV of genotype 2a (NCBI Protein Accession No. AAF01178.1) was cloned into pcDNA3.1 and then the resulting pcDNA J6dC-E2 expression vector was used. For preparation of HCVpp having envelope proteins of genotype 1a, a nucleic acid encoding the 132^{nd} to the 746^{th} amino acid residues (a part of the core protein, the E1 protein, and the E2 protein) of the protein of H77 of HCV of genotype 1a (NCBI Protein Accession No. AAB67036.1) was cloned into pcDNA3.1, and then the resulting pcDNA H77dC-E2 expression vector was used. For preparation of HCVpp having envelope proteins of genotype 1b, a nucleic acid encoding the 132^{nd} to the 747^{th} amino acid residues (a part of the core protein, the E1 protein, and the E2 protein) of the protein of TH of HCV of genotype 1b (Wakita, T. et al., J. Biol. Chem., 269: 14205-14210, 1994, Moradpour, D. et al., Biochem. Biophys. Res. Commun., 246: 920-924, 1998, and International Patent Publication WO2006/022422) was cloned into pcDNA3.1, and then the resulting pcDNA THdC-E2 expression vector was used.

Gag-Pol 5349 was used as an expression vector constructed by cloning genes encoding gag and pol of a mouse leukemia virus. Luc126 was used as a retrovirus packaging vector constructed by cloning a luciferase gene.

293T cells were subcultured in 10% FCS-DMEM medium (1% MEM nonessential amino acid solution (Invitrogen), 10 mM HEPES (pH 7.3), 1 mM sodium pyruvate, 100 unit/mL penicillin, 100 μg/mL streptomycin (Gibco: catalog No. 15140-122)) (hereinafter, referred to as “DMEM-10F”). Collagen-coated flasks (IWAKI: catalog Nos. 4100-010 and 4160-010) were used for culture. 293T cells were seeded on a collagen-coated 10-cm dish (IWAKI: catalog No. 4020-010) to 2.5×10⁶ cells/dish, and then cultured overnight. Opti-MEM (Gibco: catalog No. 11058), FuGENE6 (Roche: catalog No. 11814443001), and 3 types of construct (HCV envelope protein expression vector, Gag-Pol 5349, and Luc126) were mixed in the following amounts. Specifically, 500 μL of Opti-MEM, 21.6 μl of FuGENE6, 1 μg of HCV envelope protein expression vector, 3.1 μg of Gag-Pol 5349, and 3.1 μg of Luc126 (or mixed with the same composition ratio) were mixed, and then incubation was performed at room temperature for 15 minutes. The medium for 293T cells was exchanged with Opti-MEM (7.5 mL), a DNA complex was added to the medium, and then incubation was performed at 37° C. and 5% CO₂ for 6 hours. After completion of the reaction, washing was performed once with PBS, 8 mL of DMEM-10F was added, and then incubation was performed at 37° C. and 5% CO₂ for 48 hours. After completion of the culture, supernatants were collected and then filtered with a 0.45-μm filter, so that HCVpp solutions were obtained. HCVpp (1 mL) was dispensed and then stored at −80° C.

The thus obtained, pseudo HCV particles having structural proteins of the J6CF strain of genotype 2a were referred to as “J6CF HCVpp”, pseudo HCV particles having structural proteins of the H77 strain of genotype 1a were referred to as “1177 HCVpp”, and pseudo HCV particles having structural proteins of the TH strain of genotype 1b were referred to as “TH HCVpp”.

### 2) Measurement of Activity of Inhibiting HCV Infection

The serum samples obtained via blood collection from mice immunized via administration of each vaccine composition as in “1. Administration of vaccine composition” above were subjected to measurement of the activity of inhibiting HCV infection by the following method.

### (A) Activity of Inhibiting HCV Infection Using HCVpp Virus

Huh7.5.1 cells were seeded on 96-well plates (coated with poly-D-lysin) at 1×10⁴ cells/well, and then cultured overnight. A J6CF HCVpp virus solution (culture supernatant) obtained by the step 1) above was mixed with mouse serum in an amount equivalent thereto, and then incubated at room temperature for 30 minutes. Mouse serum was diluted 50-fold with DMEM medium (DMEM containing 10% FCS (Invitrogen), 1% MEM nonessential amino acid solution (Invitrogen), 10 mM HEPES-Tris (pH 7.3), and 1 mM sodium pyruvate) and then used (the final dilution rate for mouse serum: 100 fold). Medium for cells was discarded, and then mouse serum was added for incubation. The thus incubated virus solution was added at 50 μL/well and then incubated at 37° C. for 3 hours. The virus solution was discarded, wells were washed once with PBS at 100 μL/well, DMEM medium was added at 200 μL/well, and then incubation was performed at 37° C. for 72 hours. After medium was discarded, 25 μL/well DMEM medium and 25 μL/well Steady-Glo (Promega: catalog No. E2520) were added. Cells were lysed according to instructions included with Steady-Glo. The cell lysis solution (40 μL/well) was transferred to a white 96-well plate (Sumitomo Bakelite Co., Ltd.: catalog No. MS-8496W) and then luminescence intensity was measured using ARVO X4 (PerkinElmer).

Furthermore, to confirm whether or not serum samples derived from mice immunized with J6/JFH1-HCV particles having structural proteins of genotype 2a exhibited the activity of inhibiting HCV infection against HCV having structural proteins of genotype 1a and 1b, the activity of inhibiting HCV infection in mouse serum was measured by the method similar to the above using H77 HCVpp (pseudo HCV particles having structural proteins of genotype 1a) and TH HCVpp (pseudo HCV particles having structural proteins of genotype 1b).

### (B) Activity of Inhibiting HCV Infection Using HCVcc Virus

Huh7.5.1 cells were seeded on 96-well plates (coated with poly-D-lysin) at 1×10⁴ cells/well, and then cultured overnight. A J6/JFH1-HCV particle solution (a concentrated solution of the culture supernatant containing HCV particles; hereinafter, referred to as “J6/JFH1 HCVcc”) obtained by the step 1) in Example 2 above was used as an HCVcc virus solution. The solution was mixed with mouse serum in an amount equivalent thereto, and then incubated at room temperature for 30 minutes. Mouse serum was diluted 50-fold with DMEM medium (DMEM containing 10% FCS (Invitrogen), 1% MEM nonessential amino acid solution (Invitrogen), 10 mM HEPES-Tris (pH 7.3), and 1 mM sodium pyruvate) and then used (the final dilution rate for mouse serum: 100 fold). Medium for cells was discarded, and then mouse serum was added for incubation. The thus incubated virus solution was added at 50 μL/well and then incubated at 37° C. for 3 hours. The virus solution was discarded, wells were washed once with PBS at 100 μL/well, DMEM medium was added at 200 μl/well, and then incubation was performed at 37° C. for 72 hours. After the culture supernatant was removed, the plate was soaked in ice-cold methanol to fix the cells. Thereafter, methanol was removed via air drying, and then Block Ace (registered trademark) (Dainippon Pharmaceutical Co., Ltd.) containing 0.3% Triton (registered trademark)-X 100 (GE Healthcare) was added at 100 μL/well, thereby performing blocking overnight at 4° C. Washing was performed twice with PBS 150 μL/well, HRP-labeled anti-HCV-core antibody diluted 100-fold (Ortho Clinical Diagnostics) was added at 50 μL/well, followed by 1 hour of reaction. Washing was then performed 4 times with PBS 150 μL/well, a QuantaBlu (registered trademark) (PIERCE) reaction solution was added at 50 μL/well, the resultant was left to stand at room temperature for 30 minutes, and then a QuantaBlu stop solution was added at 50 μL/well to stop the reaction. The resultant (20 μL/well) was transferred to a black 384-well plate (Corning: catalog No. 3676) and then fluorescence intensity was measured using ARVO X4 (PerkinElmer).

Furthermore, to confirm whether or not serum samples derived from mice immunized with J6/JFH1-HCV particles having structural proteins of genotype 2a had the activity of inhibiting HCV infection against HCVcc having structural proteins of genotype 1b, the activity of inhibiting HCV infection in serum was measured by the method similar to the above using a TH/JFH1-HCV particle solution (HCVcc particles having structural proteins of genotype 1b) (hereinafter, referred to as “TH/JFH1 HCVcc”). In addition, TH/JFH1 HCVcc was prepared by the method described in WO2009/131203.

### 4. Evaluation of Immune-Response-Inducing Capacity of Vaccine Composition Containing J6E2Fc Protein as Antigen

### 1) Immune-Response-Inducing Capacity of Vaccine Composition Containing J6E2Fc Protein as Antigen

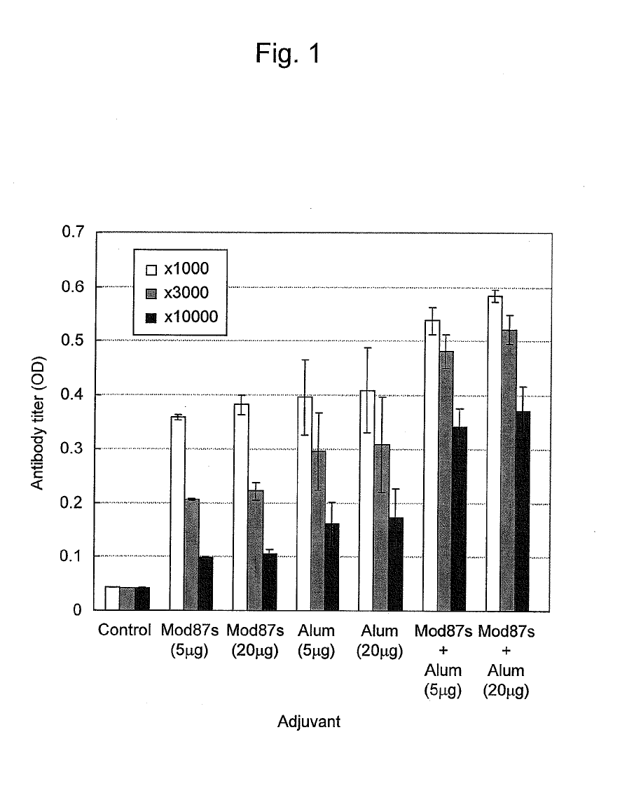

FIG. 1 shows the results of measuring antibody titers (“2. Measurement of antibody titers against E1 protein and E2 protein” above) against the E2 protein of mice to which the vaccine composition containing the J6E2Fc protein was administered as in 1) of “1. Administration of vaccine composition” above. In FIG. 1, “control” denotes the serum of a normal mouse to which no vaccine composition was administered, “5 μg or 20 μg” denotes the amounts of the J6E2Fc protein administered, and “×1000, ×3000, ×10000” denotes dilution rates for mouse serum. As shown in FIG. 1, it was confirmed that Alum+Mod87s exhibited stronger activity of inducing the antibody against the E2 protein compared with Alum or Mod87s alone.

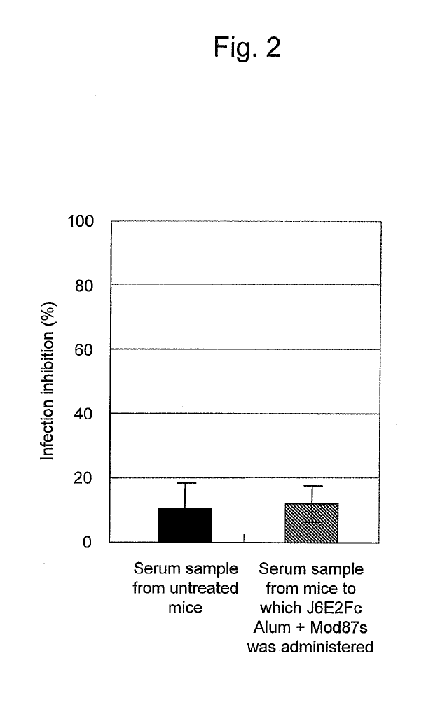

Also, from among the results of measuring neutralization titer (inhibition (%) of infection) (“3. Measurement of neutralization titer” above) for mice to which the vaccine composition containing the J6E2Fc protein was administered as in 1) of “1. Administration of vaccine composition,” FIG. 2 shows the results for mice to which the vaccine composition containing 20 μg of the J6E2Fc protein (which exhibited the highest antibody titer against the E2 protein) and Alum+Mod87s as an adjuvant was administered. As shown in FIG. 2, the serum of the immunized mice merely exhibited the inhibition (12%) of HCV infection against J6CF HCVpp.

Therefore, it was demonstrated that antibody titer against the E2 protein of the vaccine composition containing the J6E2Fc protein as an antigen is increased, even when Alum+Mod87s had been used as an adjuvant, but no activity of inhibiting HCV infection is observed.

### 2) Immune-Response-Inducing Capacity of Vaccine Composition Containing Inactivated J6/JFH1-HCV Particle as Antigen

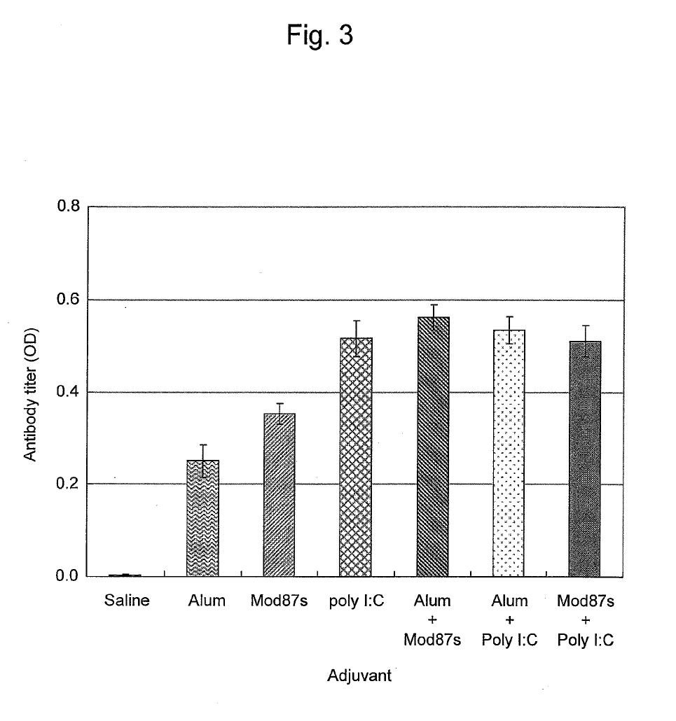

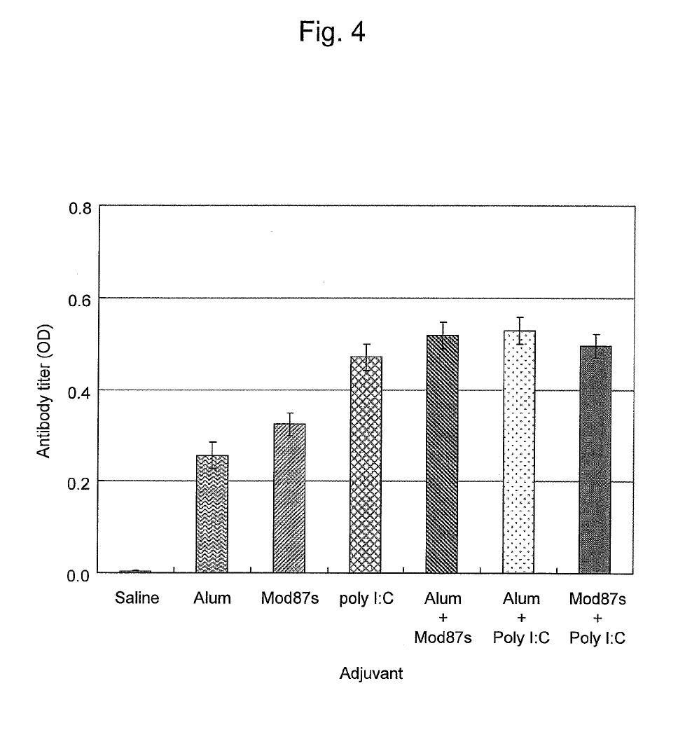

FIG. 3 and FIG. 4 show the results of measuring antibody titer against the E1 protein or the E2 protein (“2. Measurement of antibody titers against E1 protein and E2 protein” above) for mice to which the vaccine composition containing inactivated J6/JFH1-HCV was administered as in 2) of “1. Administration of vaccine composition” above. As shown in FIG. 3, antibody titer of the vaccine composition containing polyI:C alone, Alum+Mod87s, Alum+poly I:C, or Mod87s+poly I:C against the E1 protein is increased compared with that of the vaccine composition containing Alum alone or Mod87s alone. Also, as shown in FIG. 4, antibody titer of the vaccine composition containing polyI:C alone, Alum+Mod87s, Alum+poly I:C, or Mod87s+poly I:C against the E2 protein is increased compared with that of the vaccine composition containing Alum alone or Mod87s alone.

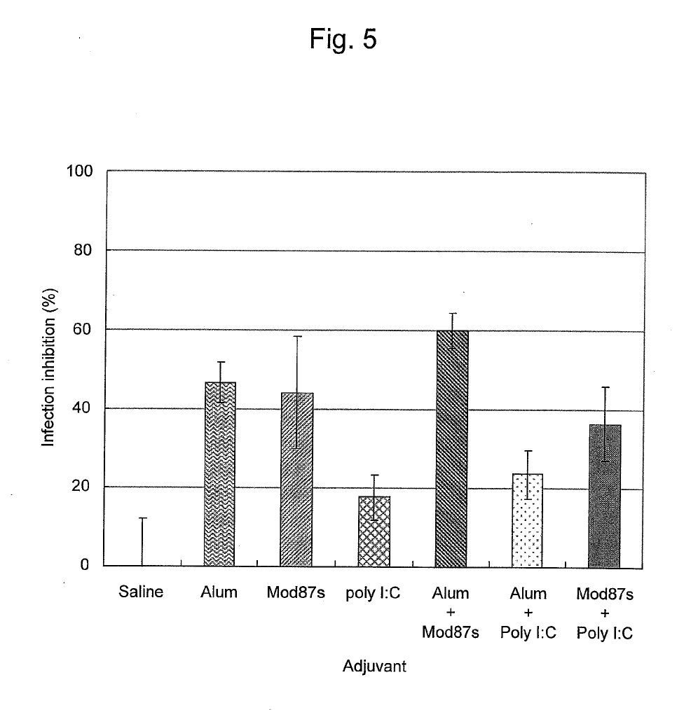

Furthermore, FIG. 5 shows the results of measuring neutralization titer (inhibition (%) of infection) (“3. Measurement of neutralization titer” above) for mice to which the vaccine composition containing inactivated J6/JFH1-HCV was administered as in 2) of “1. Administration of vaccine composition” above. As shown in FIG. 5, the vaccine composition containing a combination of Alum and Mod87s as an adjuvant exhibited the activity (60%) of inhibiting HCV infection against J6CF HCVpp. On the other hand, in the case of the other vaccine compositions containing other adjuvants, specifically, the activity (47%) of inhibiting HCV infection was merely exhibited in mice to which the vaccine composition containing Alum alone as an adjuvant (exhibited the highest activity of inhibiting HCV infection) had been administered.

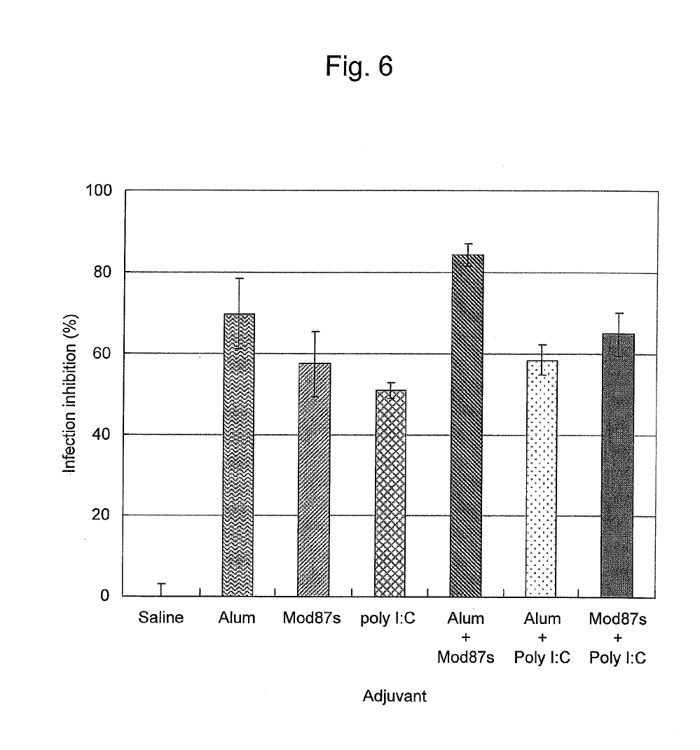

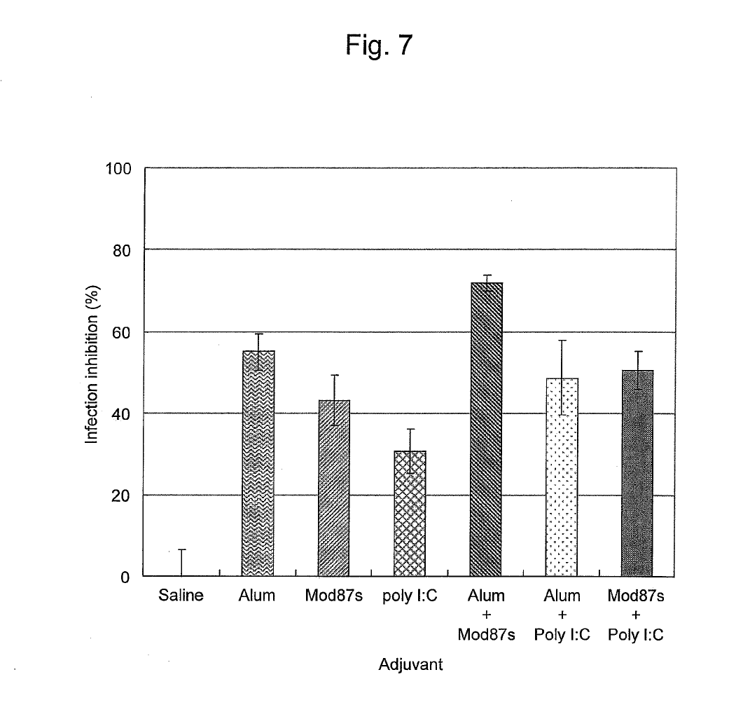

FIG. 6 (H77 HCVpp (pseudo HCV particles having structural proteins of genotype 1a)) and FIG. 7 (TH HCVpp (pseudo HCV particles having structural proteins of genotype 1b)) show the results of examining the capacity of inhibiting infection with HCV of genotype other than genotype 2a used for immunization for the serum samples of mice immunized with inactivated J6/JFH1-HCV as in 2) of “3. Measurement of neutralization titer” above. The results of measuring the activity of inhibiting HCV infection using H77 HCVpp of genotype 1a were: the activity (84%) of inhibiting HCV infection of the vaccine composition containing Alum+Mod87s as an adjuvant; and the activity (70%) of inhibiting HCV infection of the vaccine composition containing Alum alone as an adjuvant (FIG. 6). On the other hand, the results of measuring the activity of inhibiting HCV infection using TH HCVpp of genotype 1b were: the activity (72%) of inhibiting HCV infection of the vaccine composition containing Alum+Mod87s as an adjuvant; and the activity (55%) of inhibiting HCV infection of the vaccine composition containing Alum alone as an adjuvant (FIG. 7).

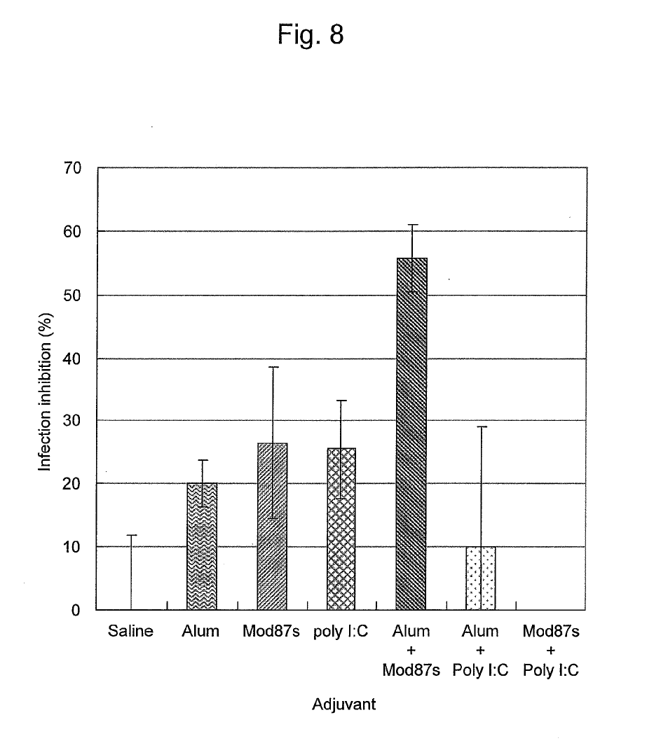

FIG. 8 shows the results of measuring neutralization titers (“3. Measurement of neutralization titers” above) using J6/JFH1 HCVcc of genotype 2a for the serum samples of mice to which the vaccine composition containing inactivated J6/JFH1-HCV was administered as in 2) of “1. Administration of vaccine composition” above. As shown in FIG. 8, the vaccine composition containing Alum+Mod87s as an adjuvant exhibited the activity (56%) of inhibiting HCV infection. On the other hand, the vaccine compositions containing other adjuvants merely exhibited inhibitory activity (26%) in mice to which the vaccine compositions containing Mod87s alone and poly I:C alone as adjuvants (exhibited the highest activity of inhibiting HCV infection) had been administered.

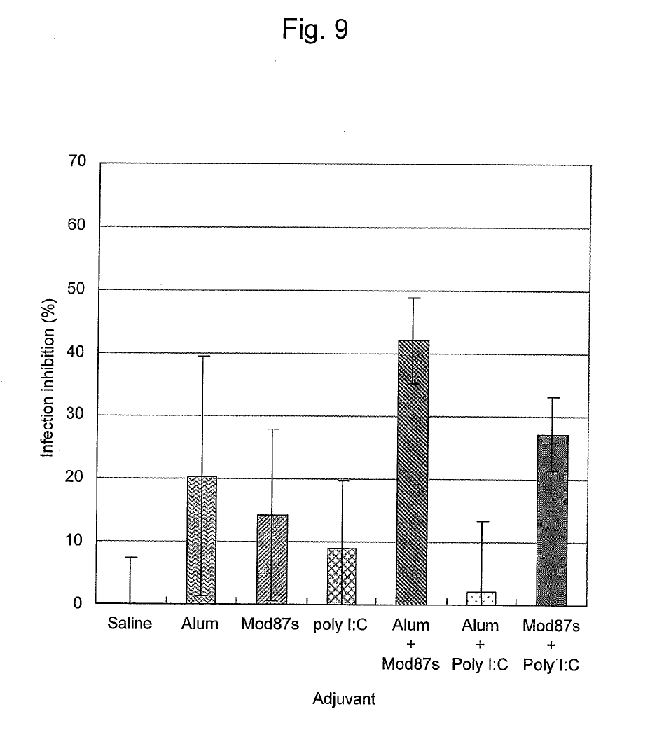

Moreover, FIG. 9 shows the results of examining the infection-inhibiting capacity of HCVcc of genotype other than genotype 2a used for immunization for the serum samples of mice to which the vaccine composition containing inactivated J6/JFH1-HCV was administered as in 2) of “3. Measurement of neutralization titer” above. As shown in FIG. 9, the results of measuring the activity of inhibiting HCV infection using TH/JFH1 HCVcc of genotype 1b were: the activity (42%) of inhibiting HCV infection in the case of the vaccine composition containing Alum+Mod87s as an adjuvant; and 27% in the case of vaccine compositions containing other adjuvants, and specifically in the case of the vaccine composition containing as an adjuvant Mod87s+poly I:C having the highest activity of inhibiting HCV infection.

These results revealed that the Alum+Mod87s adjuvant induces predominantly antibodies exhibiting the activity of inhibiting HCV infection. Moreover, these results demonstrate that among antibodies to be induced by immunization with HCV of genotype 2a, not only an antibody inhibiting infection with HCV of genotype 2a, but also an antibody inhibiting infection with HCV of genotype 1a and 1b is present.

### Sequence Listing Free Text

### SEQ ID NO: 1: JFH1

SEQ ID NO: 2: synthetic DNA J6/JFH1
SEQ ID NO: 3: synthetic DNA TH/JFH1
SEQ ID NO: 4: synthetic oligonucleotide Mod87
SEQ ID NO: 5: synthetic oligonucleotide Mod87s. 1st to 7th and 20th bases are phosphorothioated guanines.
SEQ ID NO: 6: primer J6E2Fc-s
SEQ ID NO: 7: primer J6E2Fc-as
SEQ ID NO: 8: primer J6E2dTM-as
Sequence Listing

1. A hepatitis C virus vaccine composition, comprising:
inactivated viral particles obtained by inactivating infectious hepatitis C virus particles prepared from hepatitis C virus genome that contains sequences encoding NS3 protein, NS4A protein, NS4B protein, NS5A protein and NS5B protein derived from hepatitis C virus JFH1 strain;
an oligonucleotide containing unmethylated CpG, according to SEQ ID NO: 5 in the sequence listing; and
aluminium hydroxide.

2. The hepatitis C virus vaccine composition according to claim 1, wherein the hepatitis C virus genome contains sequences encoding core protein, E1 protein, E2 protein and p7 protein derived from hepatitis C virus J6CF strain.

3. The hepatitis C virus vaccine composition according to claim 1 or 2, wherein the hepatitis C virus genome contains:
a sequence that encodes 16 amino acid residues from the N-terminal amino acid residue of NS2 protein derived from hepatitis C virus J6CF strain; and
a sequence that encodes a portion ranging from the 17th amino acid residue from the N terminus to the C-terminal amino acid residue of NS2 protein derived from hepatitis C virus JFH1 strain.

4. A hepatitis C virus vaccine composition, comprising:
inactivated viral particles obtained by inactivating infectious hepatitis C virus particles containing NS3 protein, NS4A protein, NS4B protein, NS5A protein and NS5B protein derived from hepatitis C virus JFH1 strain;
an oligonucleotide containing unmethylated CpG, according to SEQ ID NO: 5 in the sequence listing; and
aluminium hydroxide.

5. The hepatitis C virus vaccine composition according to claim 3, wherein the infectious hepatitis C virus contains core protein, E1 protein, E2 protein and p7 protein derived from the hepatitis C virus J6CF strain.

6. The hepatitis C virus vaccine composition according to claim 4, wherein the infectious hepatitis C virus is characterized in that the NS2 protein is a chimeric protein, 16 amino acid residues from the N-terminal amino acid residue of the NS2 protein are derived from the J6CF strain, and a portion ranging from the 17th amino acid residue from the N-terminus to the C-terminal amino acid residue of the NS2 protein is derived from the JFH1 strain.
